(19) United States
(12) Patent Application Publication
(10) Pub. No.: US 2007/0141707 A1
(43) Pub. Date: Jun. 21, 2007

(54) Sponge toxins

(75) Inventors: H. Scott Roderick, Aberdeen (GB); Marcel Jaspars, Old Aberdeen (GB); Betina Platt, Aberdeen (GB); Gernot Reidel, Aberdeen (GB); Debra McLaggan, Aberdeen (GB); Kristina Sepcic, Lubljana (SI)

Correspondence Address: LICATA & TYRRELL P.C., 66 E. MAIN STREET, MARLTON, NJ, 08053, US

(73) Assignee: ABERDEEN UNIVERSITY, Aberdeen (GB)

(21) Appl. No.: 10/561,500

(22) PCT Filed: Jun. 21, 2004
(86) PCT No.: PCT/GB04/02659

§ 371 Date: Jul. 24, 2006

(30) Foreign Application Priority Data
Jun. 19, 2003 (GB) 0314340.1
Sep. 19, 2003 (GB) 0322003.5

(51) Int. Cl. C12N 15/88; C12N 15/87

(52) U.S. Cl. 435/458; 435/459

(57) ABSTRACT

The present invention relates to the use of sponge toxins, in particular polymeric 1,3-alkylpyridinium salts (poly-APS), for the reversible formation of membrane pores and a method for producing such pores.

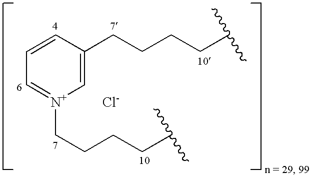

The present invention relates to the use of sponge toxins, in particular polymeric 1,3-alkylpyridinium salts (poly-APS), for the reversible formation of membrane pores and a method for producing such pores.

Over the last 500-700 million years sessile marine sponges have developed a variety of strategies to survive changing environmental conditions, potential problems with dispersal and competition with other organisms including invasion of their structure by micro-organisms and the emergence of potential predators. In addition to being able to regenerate and take on a single cell existence, sponges have an extensive armoury of chemical defences, which prevent over-predation, facilitate establishment of a sponge colony and control colonisation of the surfaces of sponges by other marine organisms. Of the many distinct chemical weapons produced by sponges a number are alkylpyridinium salts that have interesting biological properties that may be exploited. Halitoxins (1,3-APS oligomers) were originally identified from sponges of the Haploscerid genera such as Haliclona, Amphimedon and Callyspongia. Diverse biological activities have been identified for different halitoxin preparations. These include cytotoxicity, neurotoxicity and inhibition of action potentials, stimulation of transmitter release, inhibition of K⁺ conductances and anticholinesterase activity.

The chemical and biological properties of some natural pore forming halitoxins in a fraction with a mean molecular weight of 5 kDa have been previously isolated and characterised (Scott et al (2000) J Membrane Biol 176 119-131). These compounds, isolated from the sponge Callyspongia ridleyi, were stable in aqueous solution and it was found that this cocktail of natural halitoxins depolarised cultured sensory neurones by irreversibly forming pores permeable to cations. These irreversible pores result in irreversible damage to the cell wall which is lethal to the cell. When applied to artificial bilayers toxin-evoked channel-like events were obtained with unitary conductances between 145-2280 pS. Additionally, the halitoxins could release Ca²⁺ from intracellular stores.

Single stranded polynucleotide RNA and DNA have been shown to transverse lipid bilayers treated with Staphlococcus aureus α-toxin or α-hemolysin, which create pores of 1 to 2.5 nm diameter. These Staphlococcus aureus α-toxin or α-hemolysin produce irreversible pores. Double stranded DNA has been transferred across lipid bilayers and into intact cells using lipid-micelle-mediated transfection (e.g. lipofection), electroporation and microinjection but these approaches yield variable results and can be difficult to control often requiring specialist equipment or laborious pretreatments and optimisation.

It is therefore an object of the present invention to attempt to overcome the disadvantages of the prior art.

According to the present invention, there is provided use of a composition comprising a sponge toxin for the reversible formation of a membrane pore.

It has now been established that a pore can be reversibly formed using a sponge toxin (i.e a pore can be formed and then the pore collapses allowing the cell membrane integrity to be re-established). The term “pore” has been used to describe the openings in the cell membranes produced by the sponge toxin. This seems appropriate when considering the transient changes in conductance observed, and channel-like events in artificial lipid bilayers. The advantage of reversibly producing a pore is that the pore can be used to introduce material, (e.g DNA or drugs) to a cell without permanently damaging the cell membrane, which can be lethal to the cell. This transient and stable transfection has not been shown by the prior art. The advantage of sponge toxins are that they are simple to use, highly chemically stable and have good water solubility. In this connection, in contrast to some methods of the prior art there is no need to encapsulate DNA in liposomes before transfection.

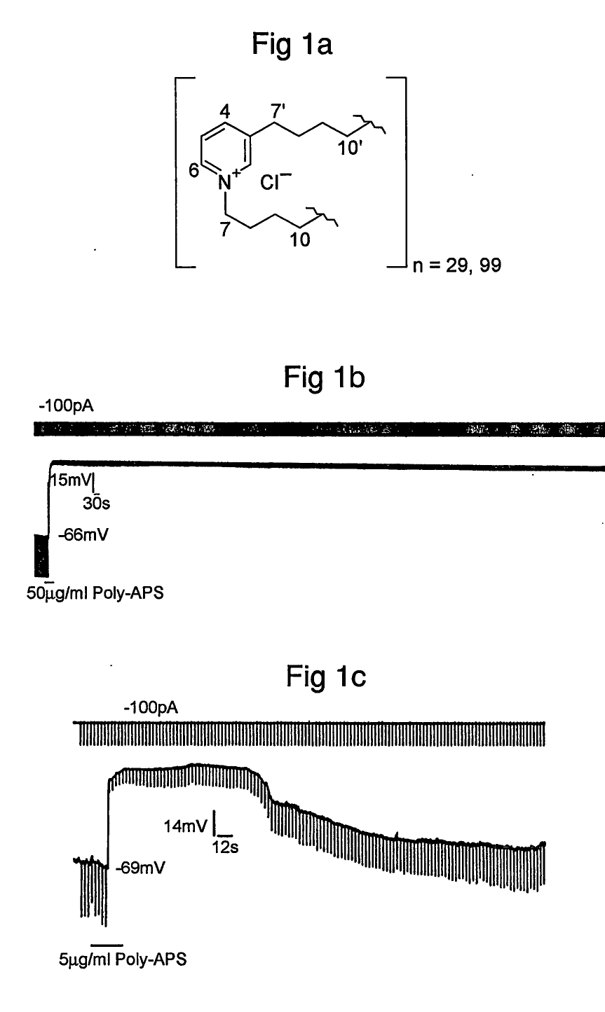

Preferably, the sponge toxin comprises at least one polymeric 1,3-alkylpyridinium salt (poly-APS). The halitoxin preparation mentioned herein is composed of many (tens) of distinct polymeric 1,3-alkypyridinium compounds. This preparation has a mean molecular weight of about 5 kDa. In contrast, the poly-APS preparation is composed of two polymeric 1,3-alkylpyridinium compounds with 29 and 99 repeating monomeric units as set out in FIG. 1a.

Poly-APS has been shown in aqueous solutions to form non-covalently bound aggregrates with a mean hydrodynamic radius of 23±2 nm. The radius of pores formed within these aggregates has been estimated on bovine erythrocytes by the use of osmotic protectants, and calculated with the Renkin equation to be about 2.9 nm. The combination of the pore sizes and the transient nature of the pore formation means that poly-APS is a preferable means of forming the reversible membrane pores, especially when macromolecules such as DNA are to be transported across the cell membrane.

Preferably, the sponge toxin is obtained from the sponge Reniera sarai, Callyspongia ridleyi, Haliclona erina, Haliclona rubens, Haliclona viridis, Amphimedon viridis, Callyspongia fibrosa and Amphimedon compressa.

Large numbers of sponge species produce pore-forming molecules. Halitoxin compounds have been isolated from a number of Haplosclerid genera.

Preferably, the sponge toxin has a molecular weight of between substantially 5 kDa and 20 kDa. Further preferably, the sponge toxin has a molecular weight of 5.5 kDa or 18.9 kDa. In a preferred embodiment, the composition comprises more than one sponge toxin each with a different molecular weight.

Conveniently the sponge toxin is formed from between 1 and 150 monomeric units as set out in FIG. 1a. Preferably the sponge toxin is formed from between 20 and 100 monomeric units. In a preferred embodiment the sponge toxin is formed from 29 or 99 monomeric units.

Preferably, the concentration of sponge toxin is between 0.5 ng/ml and 5 μg/ml. Further preferably, the concentration of sponge toxin is between 0.5 ng/ml and 1 μg/ml. In a preferred embodiment the concentration of sponge toxin is between 0.5 ng/ml and 0.5 μg/ml.

According to a further aspect of the present invention, there is provided a method for the reversible formation of a membrane pore, the method comprising the steps of:—

a) incubating the membrane in the presence of a composition comprising sponge toxins; and

b) removing the composition from contact with the membrane.

This method of the present invention can be used in in vitro and in vivo techniques. In in vitro techniques, the present invention can be used as a biochemical research tool such as to study the transfection of cells with macromolecules such as proteins, DNA including plasmid cDNA, peptides, lipids, oligonucleotides and membrane impermeable such as fluorescent molecules (e.g. Lucifer yellow, neurobiotin, biocytins and dextran conjugates) and in vivo the method of the present invention can be used to transfer DNA to cells for gene therapy, in addition to the transfer of proteins, peptides, lipids and oligonucleotides.

Conveniently, a zinc, mercury, nickel or cobalt solution is used to attenuate the reversible formation of a membrane pore. Preferably, the concentration of the zinc solution is between substantially 1 to 2 mM. In preferred embodiments the concentration of zinc is 1.5 mM.

DNA, Serum Albumin and Cholesterol can also be used to Attenuate or Prevent Pore Formation.

According to a yet further aspect of the present invention, there is provided a method for transfection of DNA into a cell in vitro, the method comprising the steps of:—

a) incubating the cell in the presence of a composition comprising a sponge toxin;

b) removing the composition from contact with the membrane; and

c) adding nucleic acid.

The transfection of nucleic acid (e.g. DNA such as cDNA, RNA such as mRNA) into a cell in a stable manner is important as a biochemical research tool.

Preferably, the cell is incubated in the presence of composition for between 1 and 20 minutes prior to addition of nucleic acid. Further preferably, the cell is incubated in the presence of the composition for 5 minutes prior to the addition of nucleic acid.

It has been found that incubation for these periods results in an acceptable transfection rate with minimum disruption of the cell membrane.

Preferably, between 1.0 and 5.0 μg nucleic acid is added. Further preferably, 2.5 μg nucleic acid is added.

It has been found that the addition of these amounts of DNA result in an acceptable transfection rate. In this connection, an acceptable transfection rate is considered to be in the order of 20%.

Further preferably, the method comprises the additional steps of:—incubating the cell, in the presence of the composition and nucleic acid; and replacing the composition and DNA with a standard cell media.

Preferably, the cell is incubated in the presence of the composition and nucleic acid for between substantially 20 and 200 minutes. Preferably, the incubation time is 180 minutes.

According to a further aspect of the present invention there is provided a method for transfection of a macromolecule into a cell in vivo, the method comprising the step of:—

a) incubating the cell in the presence of a composition comprising a sponge toxin and the macromolecule.

Preferably, the macromolecule is a protein, a peptide, a lipid, an oligonucleotide, or a nucleic acid e.g. cDNA.

Further preferably, the macromolecule is the cytoskeletal protein tau.

Preferably, the cell is a hippocampal neurone.

According to a yet further aspect of the present invention, there is provided a model for use in the study of neurological disease or treatments thereof, the model comprising a rodent having undergone application of a composition comprising a sponge toxin, tau protein and phosphatase inhibitor to the hippocampus.

Preferably, the neurological disease is Alzheimer's.

Preferably, the rodent is a rat or a mouse.

According to further aspect of the present invention there is provided a method of studying a neurological disease, the method comprising:

a) applying a composition comprising a sponge toxin, tau protein and phosphatase inhibitor to the hippocampus of a rodent; and

b) studying the effect on the rodent.

Preferably, the phosphatase inhibitor is okadaic acid.

The invention will now be described in further detail with reference to the accompanying figures and experimental data, in which:—

FIG. 1A shows the chemical structure of poly-APS from the marine sponge Reniera sarai. The preparation used was a mixture of polymers composed of repeating units (29 and 99 units).

FIG. 1B shows the irreversible actions of poly-APS (50 μg/ml) on membrane potential and electrotonic potentials evoked by 100 pA hyperpolarising current commands.

FIG. 1C shows the reversible actions of poly-APS (5 μg/ml) on membrane potential and electrotonic potentials (used to calculate input resistance). Records were obtained from cultured DRG neurones.

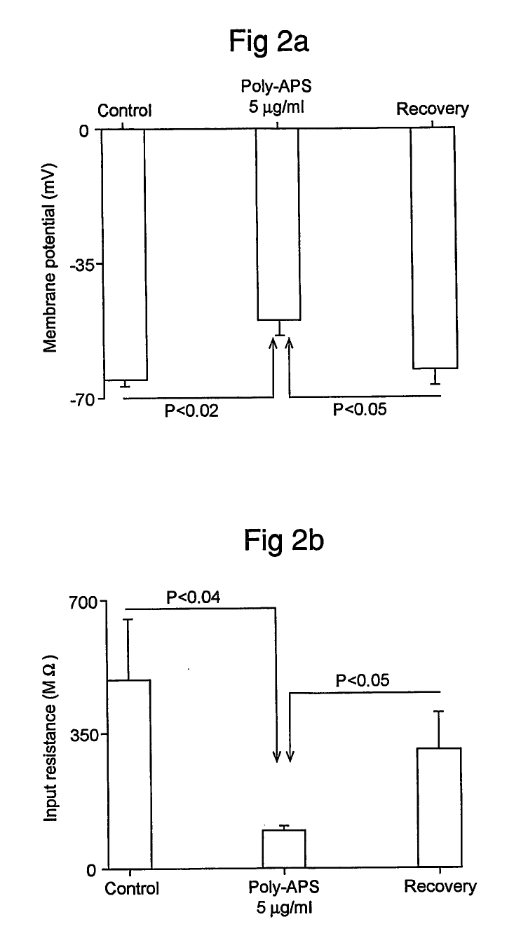

FIG. 2A shows a bar chart showing the reversible actions of 5 μg/ml poly-APS on membrane potential (n=8). Poly-APS was applied to cultured DRG neurones for 20 seconds.

FIG. 2B shows a bar chart showing the reversible actions of 5 μg/ml poly-APS on input resistance (n=8). Poly-APS was applied to cultured DRG neurones for 20 seconds.

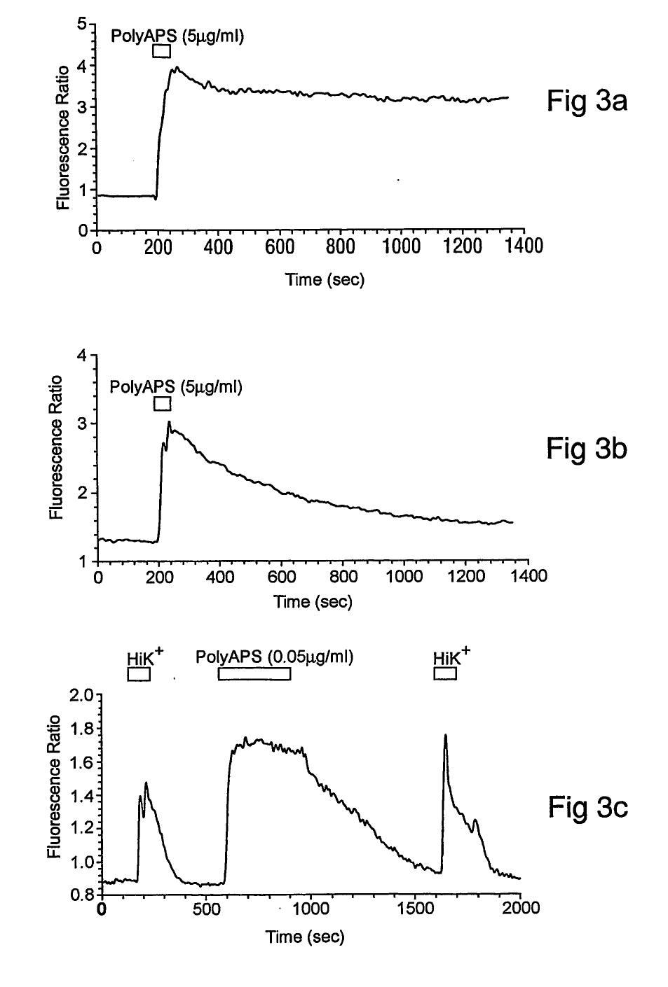

FIG. 3A shows a record of an irreversible rise in intracellular Ca²⁺-evoked by 5 μg/ml poly-APS. These recordings are from a heterogeneous population of cells and so responses vary.

FIG. 3B shows a record from a neurone in the same preparation as in FIG. 3A showing a reversible rise in intracellular Ca²⁺-evoked by 5 μg/ml poly-APS.

FIG. 3C shows a trace from a single neurone showing the reversible action of 0.05 μg/ml poly-APS.

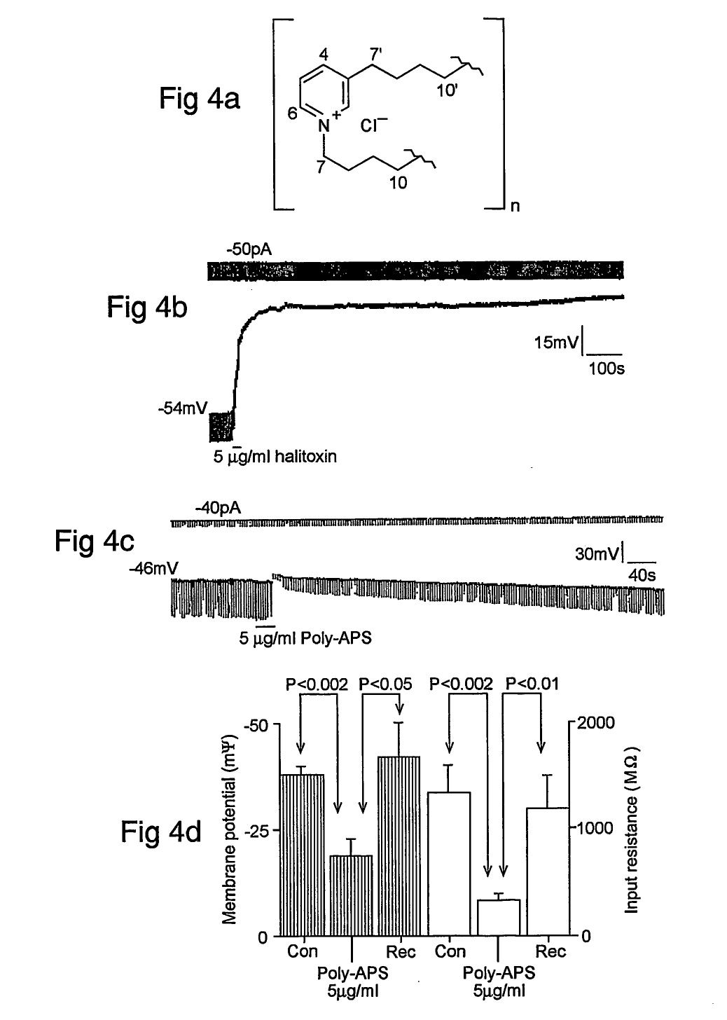

FIG. 4A shows the structure of halitoxin.

FIG. 4B shows a record showing the irreversible collapse of membrane potential and input resistance evoked by 20 s application of 5 μg/ml halitoxin. Top trace 50 μA hyperpolarising current commands evoked every 3 s, bottom trace showing the membrane potential and electrotonic potentials.

FIG. 4C shows a record showing the reversible reductions in membrane potential and input resistance evoked by 20 s application of 5 μg/ml poly-APS. Top trace shows 40 pA hyperpolarising current commands evoked every 3 s, bottom trace shows the membrane potential and electrotonic potentials.

FIG. 4D shows a bar graph showing the effects of poly-APS (5 μg/ml) on membrane potential (filled bars) and input resistance (open bars), mean values are presented under control conditions (con; n=9), in the presence of toxin (n=9) and at 10 minutes recovery (rec; n=6).

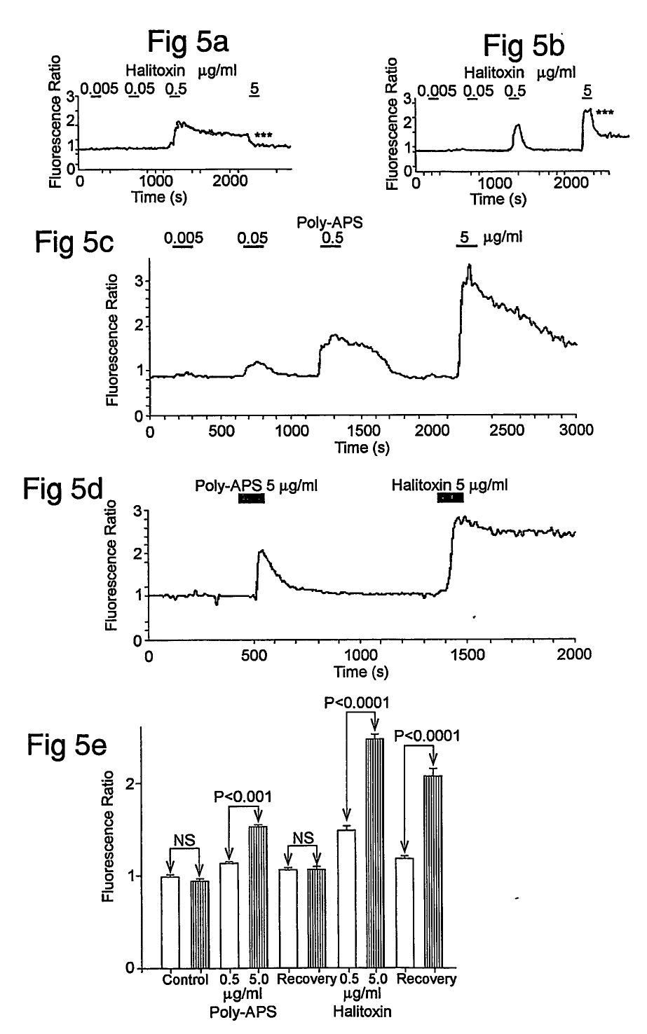

FIG. 5A shows a trace showing a single sustained response to the action of halitoxin preparations on intracellular Ca²⁺ in HEK293 cells loaded with fura-2. No responses were obtained with 0.005 and 0.05 μg/ml halitoxin and *** denote the points of dye loss and cell damage produced by 5 μg/ml halitoxin.

FIG. 5B shows a trace showing a transient response to 0.5 μg/ml halitoxin. No responses were obtained with 0.005 and 0.05 μg/ml halitoxin and *** denote the points of dye loss and cell damage produced by 5 μg/ml halitoxin.

FIG. 5C shows a single example trace showing the dose-dependent actions of poly-APS on fura-2 fluorescence ratio indicative of a rise in intracellular Ca²⁺.

FIG. 5D shows a single example trace showing transient and sustained responses to poly-APS (5 μg/ml) and halitoxin (5 μg/ml) respectively.

FIG. 5E shows a bar chart showing the mean peak fluorescence values for experiments carried out to investigate the actions of 0.5 μg/ml poly-APS and halitoxin (open bars) and 5 μg/ml poly-APS and halitoxin (filled bars).

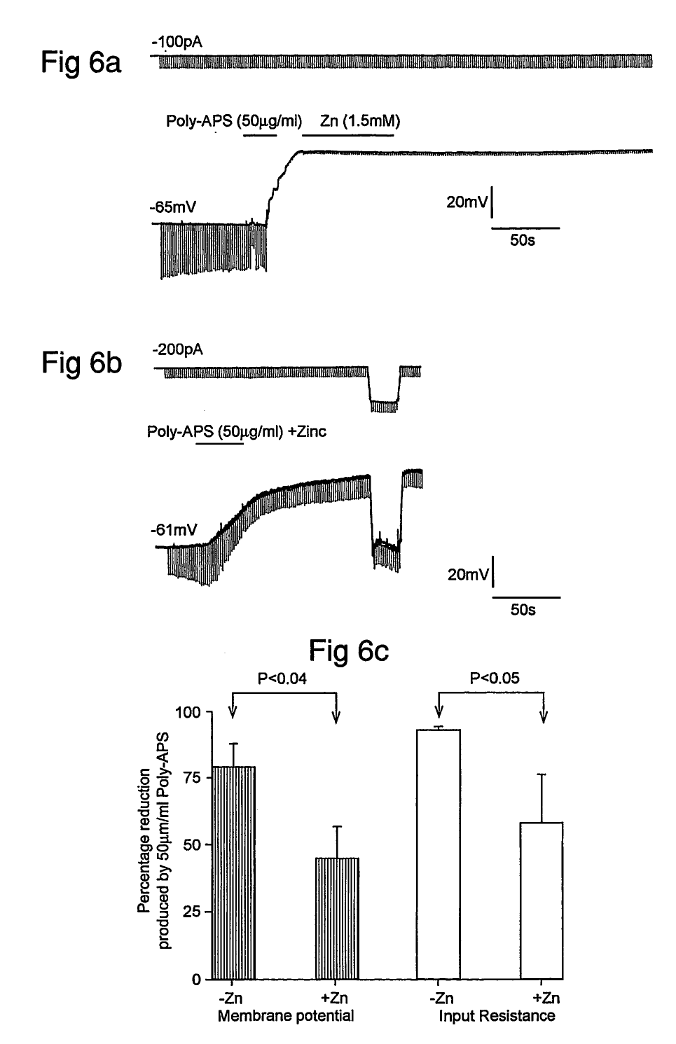

FIG. 6A shows a record showing the lack of effect of acute application of NaCl-based extracellular solution containing 1.5 mM zinc applied after poly-APS had evoked changes in membrane potential and input resistance.

FIG. 6B shows a record showing a slowed and reduced response to poly-APS after poly-APS was applied to the cells in the continual presence of NaCl-based extracellular solution containing 1.5 mM zinc. Constant current injection was applied at the end of the recording to hyperpolarise the membrane potential back to the resting level.

FIG. 6C shows a bar chart showing the significant influence of the continued presence of zinc on poly-APS-evoked changes in membrane potential (n=9 and 7) and input resistance (n=8 and 7).

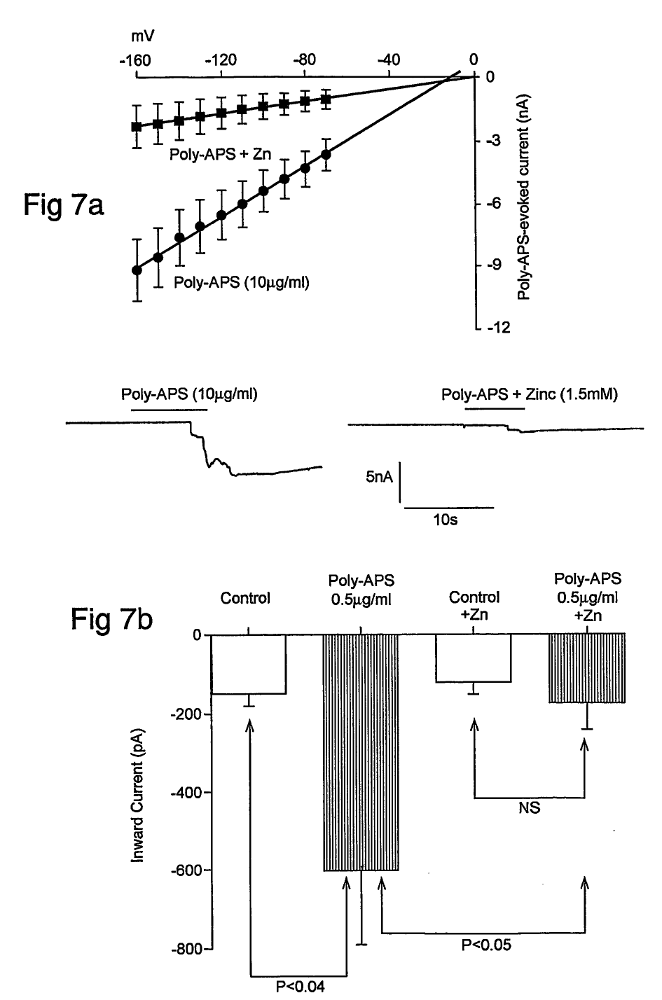

FIG. 7A shows current voltage relationships for the poly-APS evoked currents in the presence and absence of zinc. Linear I/V relationships were obtained between 160 and 70 mV after leakage subtraction (the r2 values were 0.996 and 0.995 for poly-APS applied alone and in the continual presence of zinc respectively). Inset traces show currents evoked at 90 mV by poly-APS applied in the absence and presence of zinc.

FIG. 7B shows a bar chart showing the inhibition of poly-APS (0.5 μg/ml)-evoked inward currents by zinc. Control data show the mean holding currents required to clamp the neurones at 90 mV, zinc had no significant effect on the mean holding current. Poly-APS in the absence of zinc evoked a significant inward current (n=5), however no significant inward current was produced by poly-APS in the presence of zinc (n=6).

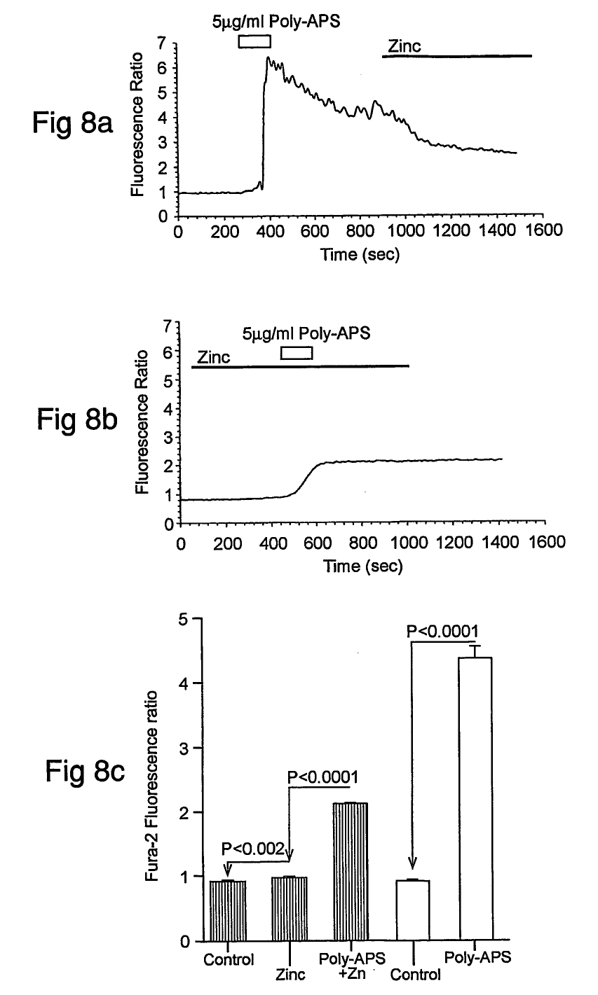

FIG. 8A shows a record showing an increase in intracellular Ca²⁺ evoked by 5 μg/ml poly-APS. Note that zinc was applied but that it was difficult to determine its actions due to the decay of the fluorescence ratio during washing. Irreversible responses were not attenuated by acute application of zinc.

FIG. 8B shows a record showing the sustained but relatively modest rise in fluorescence ratio observed when poly-APS were applied in the continual presence of zinc.

FIG. 8C shows a bar chart showing the mean imaging data derived from applying poly-APS (5 μg/ml) in the presence of zinc (filled bars, n=13) and absence of zinc (open bars, n=46). Zinc alone produced a significant but very modest increase in fluorescence ratio but subsequently attenuated the response to poly-APS.

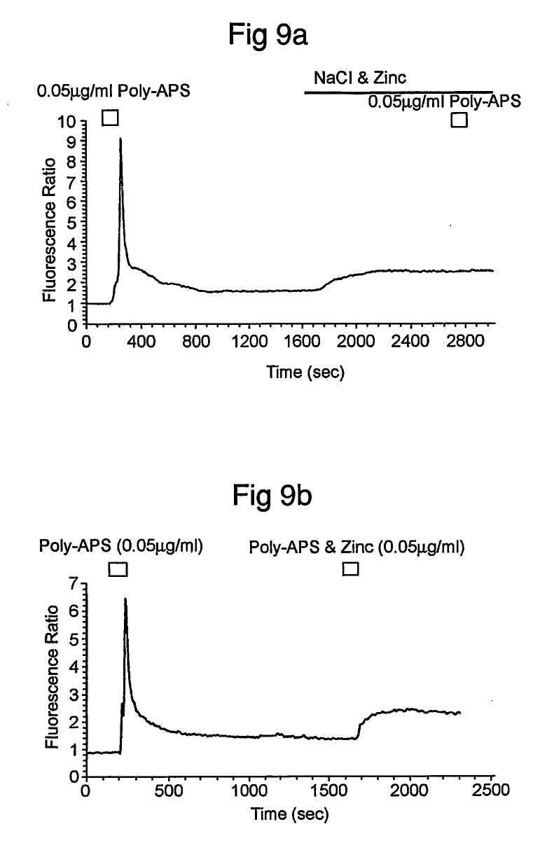

FIG. 9A shows a record of fluorescence ratio changes produced in a single DRG neurone. Poly-APS (0.05 μg/ml) evoked a transient increase in Ca²⁺; zinc was then applied and caused a sustained rise in fluorescence. However, a further application of poly-APS (0.05 μg/ml) evoked no further increase in intracellular Ca²⁺ as reflected by the stable fluorescence ratio value.

FIG. 9B shows a record of a similar experiment to that in FIG. 9A except that zinc was not pre-applied but only simultaneously applied with the second application of poly-APS (0.05 μg/ml).

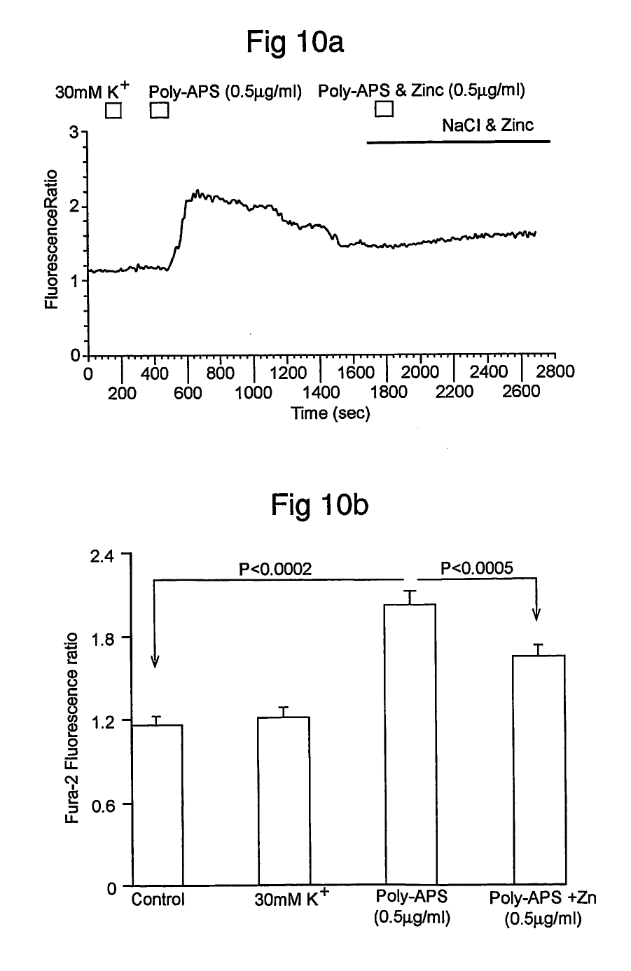

FIG. 10A shows an example record from a HEK 293 cell showing that these cells did not respond to extracellular solution containing a depolarising concentration of KCl (30 mM) and therefore did not express voltage-activated Ca²⁺ channels. However, this cell responded to the first application of poly-APS but showed only a modest increase in fluorescence ratio when poly-APS (0.5 μg/ml) was applied for a second time but with zinc present.

FIG. 10B shows a bar chart showing mean fura-2 fluorescence ratios under control conditions, during stimulation with 30 mM KCl and during application of poly-APS without and with zinc (n=11).

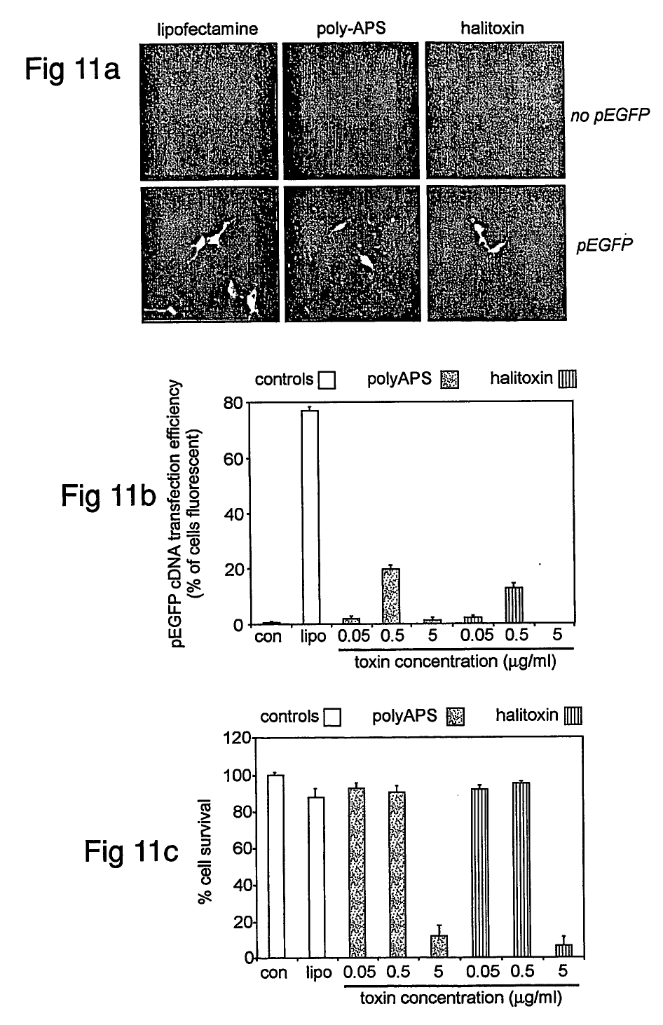

FIG. 11A shows HEK293 cells treated with lipofectamine, 0.5 μg/ml poly-APS or 0.5 μg/ml halitoxin in the absence (upper panels) or presence (lower panels) of pEGFP. Confocal images of fluorescent cells captured 48 h post-transfection, images are representative of at least 3 other independent experimental repeats.

FIG. 11B shows HEK293 cells transfected with pEGFP using either lipofectamine or 0.05 μg/ml, 0.5 μg/ml, 5.0 μg/ml poly-APS/halitoxin were assessed for percentage of total cells fluorescent 48 h post-transfection. Data shown are means±SEM, n=3.

FIG. 11C shows HEK293 cells transfected and assessed as in FIG. 11B, which were subjected to crystal violet staining 48 h post-transfection to quantify intact and adherent cells. Data expressed as % of surviving cells relative to cDNA-only control, shown as means±SEM, n=3.

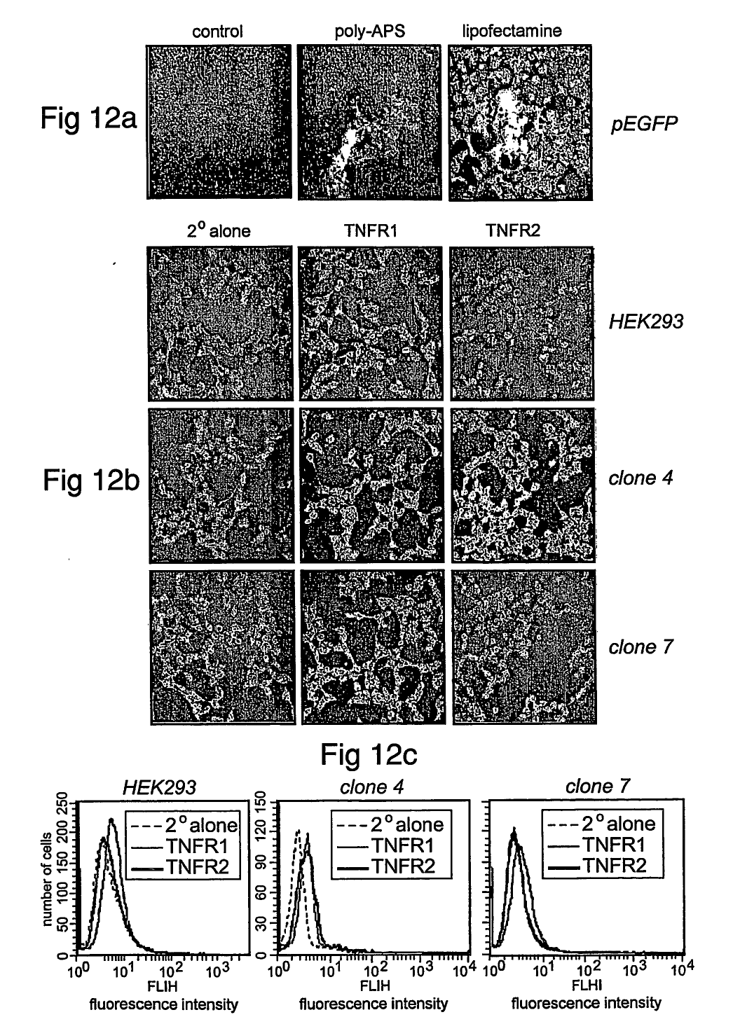

FIG. 12A shows HEK293 cells co-transfected with pEGFP and pBABE cDNA using no transfection vehicle (left-hand panel), 0.5 μg/ml poly-APS (centre panel) or lipofectamine (right hand-panel). Following hygromycin B selection, cells were imaged under confocal microscope and photographed. Photographs are representative of 3 other independent experimental repeats.

FIG. 12B shows HEK293 cells co-transfected with TNFR2 and pBABE cDNA using 0.5 μg/ml poly-APS were selected with hygromycin B and individual stable clones probed with no antibody (left-hand panels), TNFR1-(centre panels) or TNFR2-specific mouse monoclonal antibodies (right-hand panels). Subsequent secondary labelling with FITC anti-mouse IgG allowed confocal visualisation. Shown here are untransfected HEK293 cells (upper panels), a positive TNFR2 clone (clone 4) (middle panels) and a negative TNFR2 clone (clone 7) (lower panels). Photographs are representative of 3 other independent experiments.

FIG. 12C shows the results from Poly-APS transfected stable TNFR2 clones which were dissociated, probed with no antibody, TNFR1- or TNFR2-specific mouse monoclonal antibodies and secondary labelled with FITC anti-mouse IgG. Subsequent FACS analysis allowed quantitation and comparison of TNFR expression between untransfected HEK293 cells (left-hand panel), a TNFR2 positive clone (clone 4) (centre panel) and a TNFR2 negative clone (clone 7) (right-hand panel). Histograms are representative of several other repeat experiments.

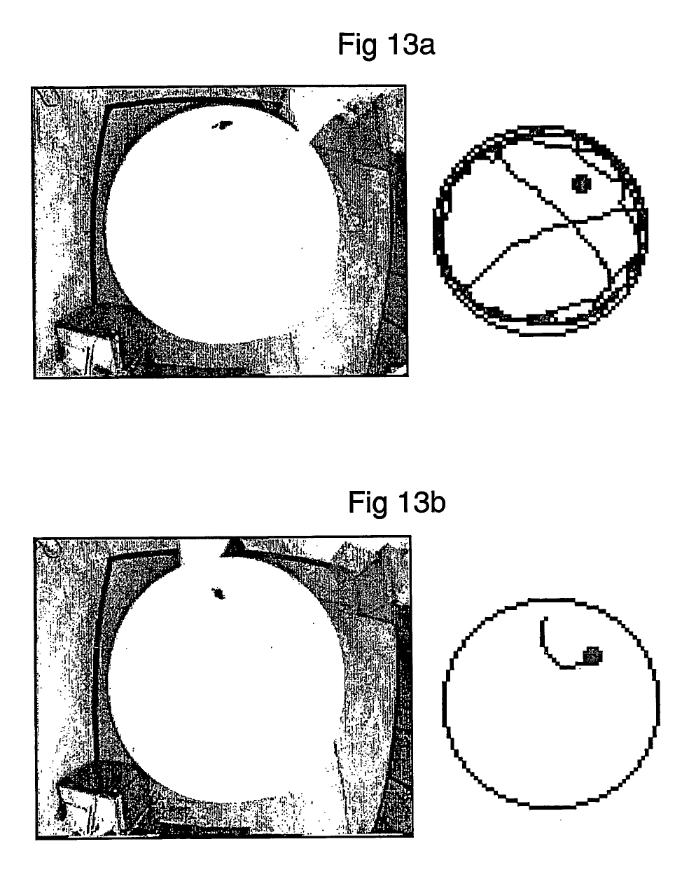

FIGS. 13A and B depict trials 1 and 16, respectively, and the swimming paths taken by the animals to the platform in the water maze experiments. Water maze experiments involve training animals to find a platform in a quadrant of a circular bath. Once the animal has learnt the position of the platform, the platform can be moved to test the animal's ability to show reversal learning. With trial 1 (FIG. 13A) the animal swims round the bath and has not learnt where the platform is, however during trial 16 (FIG. 13B) the animal swims directly to the platform.

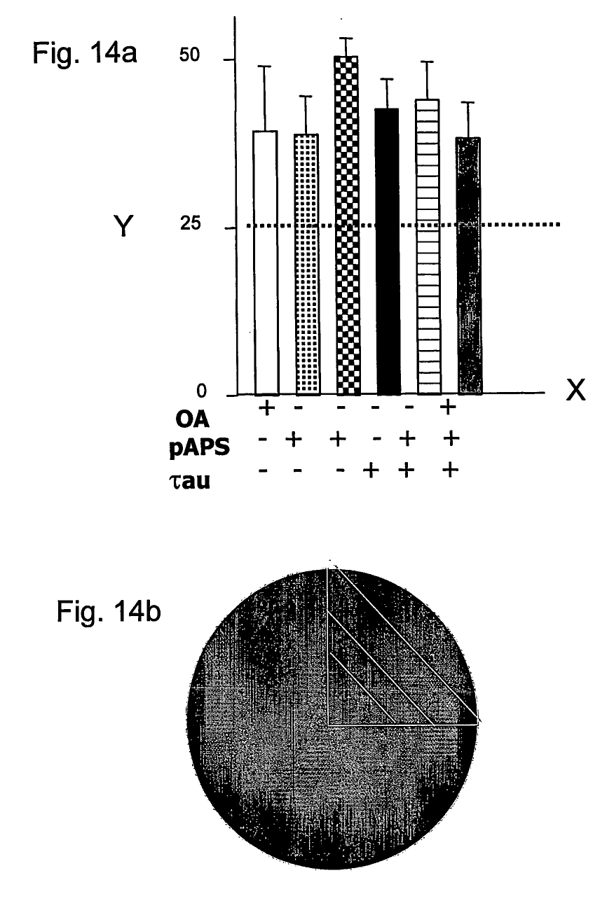

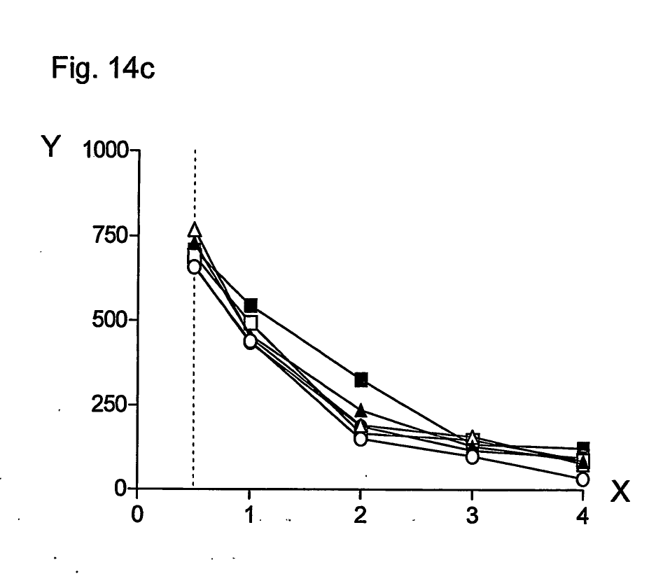

FIG. 14A shows a bar chart showing that all animals learn where the platform is in the water maze experiments, as reflected by the time (above 25%) spent in the quadrant with the platform. The Y axis represents percentage time in target quadrant.

FIG. 14B shows in which quadrant of the circular bath the platform was positioned during the water maze experiments for the above mentioned experiment identified in FIG. 14A.

FIG. 14C shows a line graph showing that all animals learn here the platform is in the water maze experiments, as reflected by the decreasing swimming path lengths over a period of 4 days. The Y axis represents the path length in centimetres and the X axis represents the number of days.

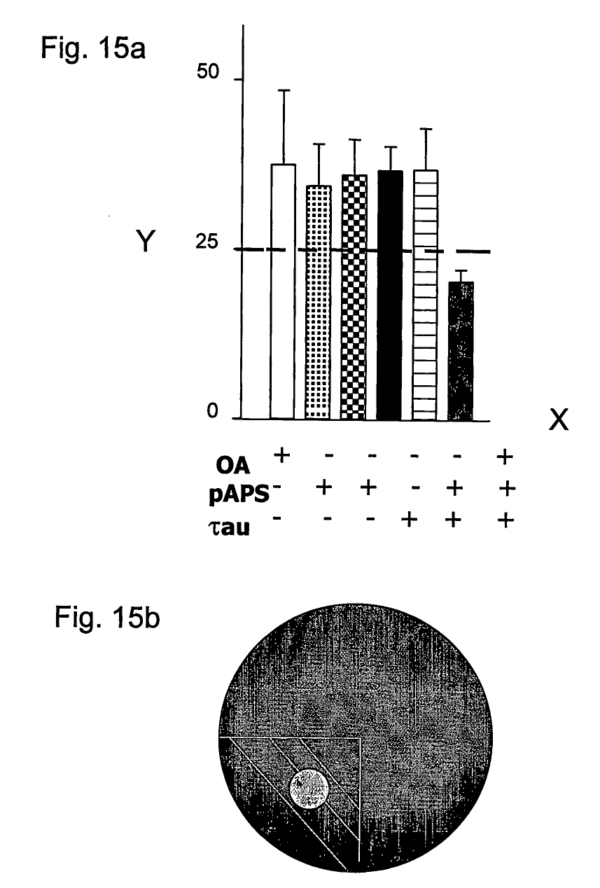

FIG. 15A shows a bar chart showing that when the platform was moved into a different quadrant of the circular bath during the water maze experiments, the animals that have been treated with poly-APS, tau and okadaic acid fail to show learning reversal, while all other treatment groups do. The Y axis represents percentage time in target quadrant.

FIG. 15B shows in which quadrant of the circular bath the platform was positioned during the water maze experiments for the above mentioned experiment identified in FIG. 15A.

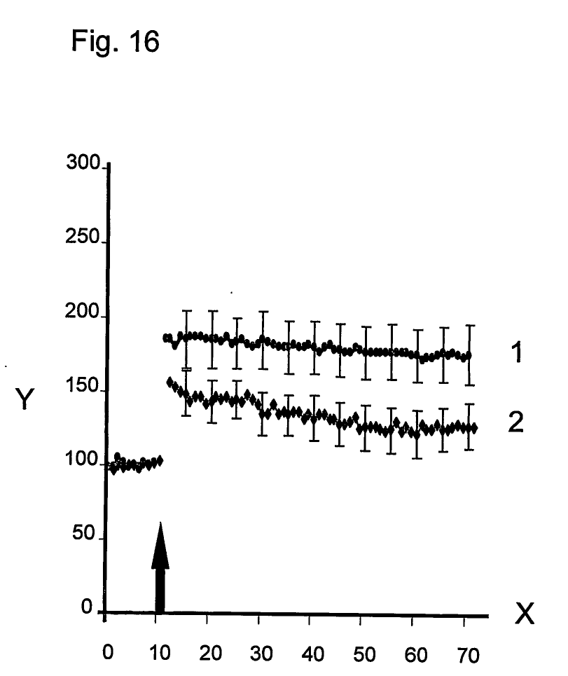

FIG. 16 shows that brains from animals treated with poly-APS, tau and okadaic acid exhibit less long term potentiation (LTP). LTP is a form of neuronal plasticity which lasts many hours or days. It is also shown that if pathways get tetanic stimulation (arrow) subsequent electrophysiological signalling is increased. The experiments were performed ex vivo on hippocampal slices. The Y axis represents the percentage Population Spike Amplitude and the X axis represents time in minutes. Data set 1 represents control data and data set 2 represents brains from animals treated with poly-APS acid, tau and okadaic.

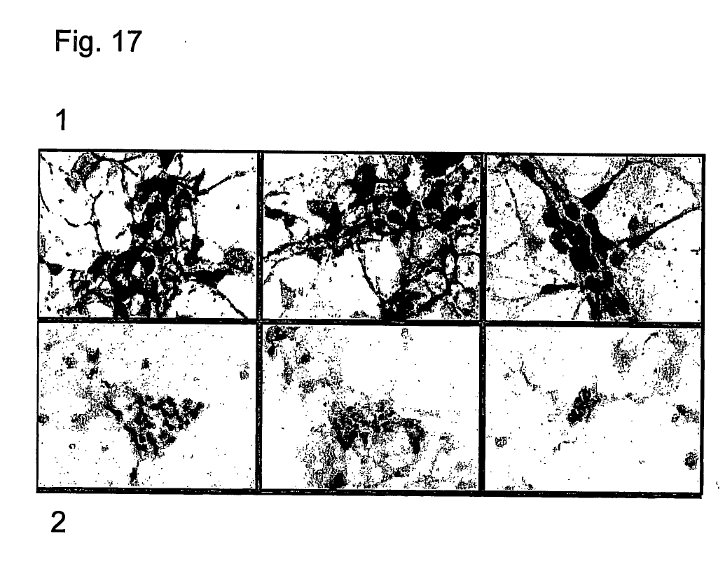

FIG. 17 shows the delivery of tau to the intracellular compartments of hippocampal neurones in culture. The tau levels (black) are detected using tau-specific antibodies. Data set 1 represents brains from animals treated with poly-PS acid, tau and okadaic and data set 2 represents control data.

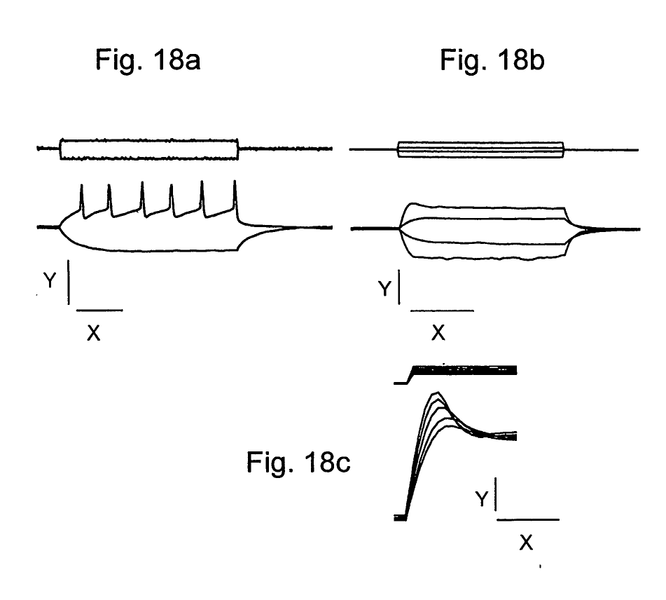

FIG. 18A shows a record of action potentials from cultured hippocampal neurones treated with poly-APS and tau. FIG. 18C shows that neurones treated with poly-APS, tau and okadaic acid fail to show action potential firing even when stimulated with much larger currents. In FIGS. 18A and 18B the Y axis represents 100 mV and the X axis represents 50 ms. In FIG. 18C the Y axis represents 25 mV and the X axis represents 10 ms.

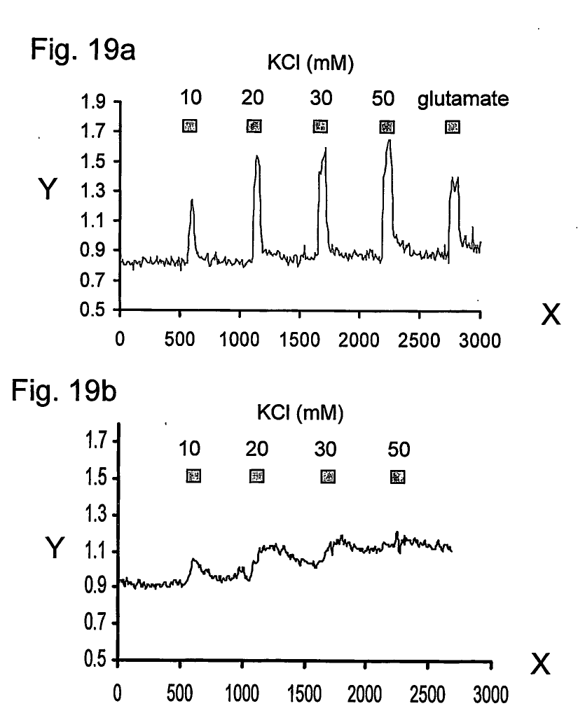

FIGS. 19A and 19B show a record from cultured hippocampal neurones stimulated with KCl pulses showing that neurones treated with poly-APS, tau and okadaic acid fail to show proper calcium transients. Calcium transients were measured with fura-2 calcium imaging. Combined with the data recorded in FIGS. 18A, 18C and 18C, this shows that the excitability of these neurones is inhibited, probably by mis-functioning of voltage-activated channels. In FIGS. 19A and 19B the Y axis represents fluorescence ratio and the X axis represents time in seconds.

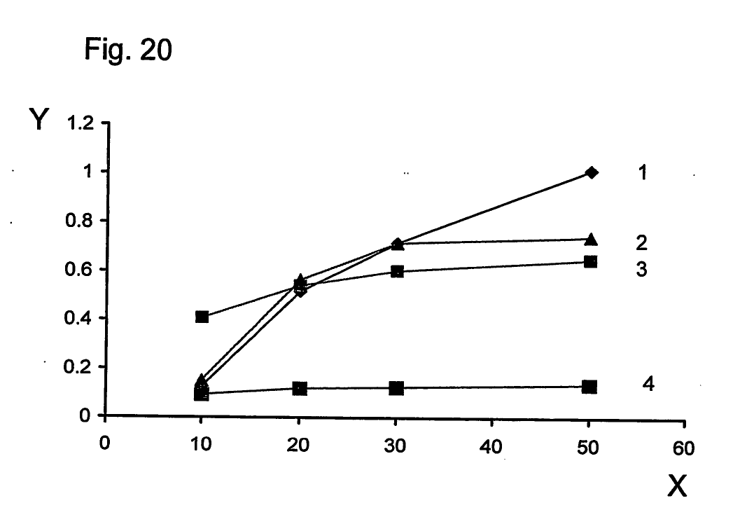

FIG. 20 shows a record of mean data for calcium transients exhibited by neurones exposed to the different treatments identified thereon. Neurones treated with poly-APS, tau and okadaic acid clearly exhibit a distinct fluorescence ratio. Combined with the data recorded in FIGS. 18 and 19, this shows that the excitability of these neurones is greatly reduced, probably involving mis-functioning of voltage-activated channels. The Y axis represents fluorescence ratio and the X axis represents KCl concentration in mM. Data set 1 is the control, data set 2 represents neurones treated with poly-APS and okadaic acid, data set 3 represents neurones treated with poly-APS and tau and data set 4 represents neurones treated with poly-APS, tau and okadaic acid.

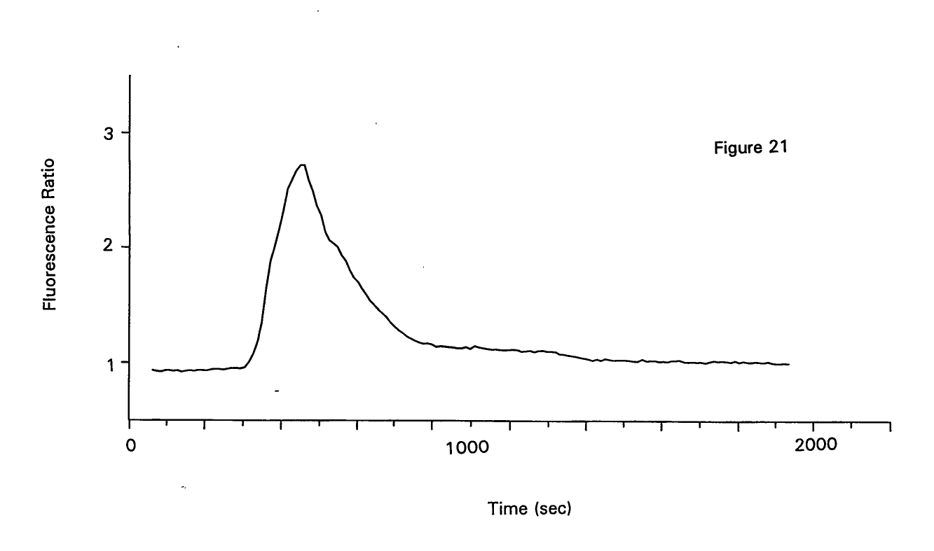

FIG. 21 shows HEK 293 cells: Ca²⁺ influx evoked by pore formation using poly-APS 1.0 μM at 12° C.

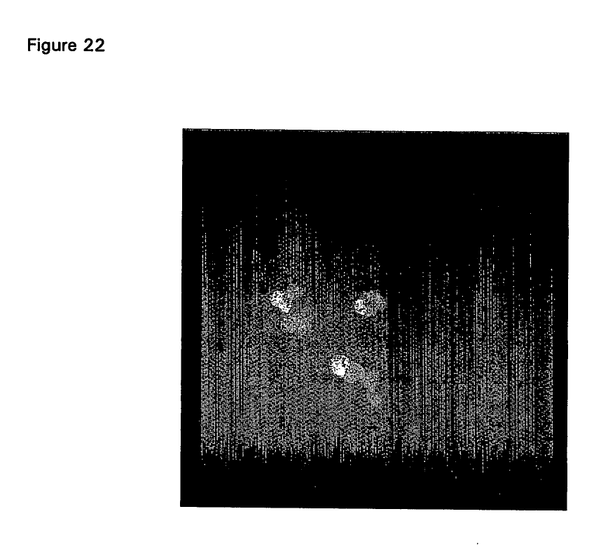

FIG. 22 shows HEK 293 cells: Transfection with cDNA for enhanced fluorescent protein can be achieved at 12° C. using poly-APS.

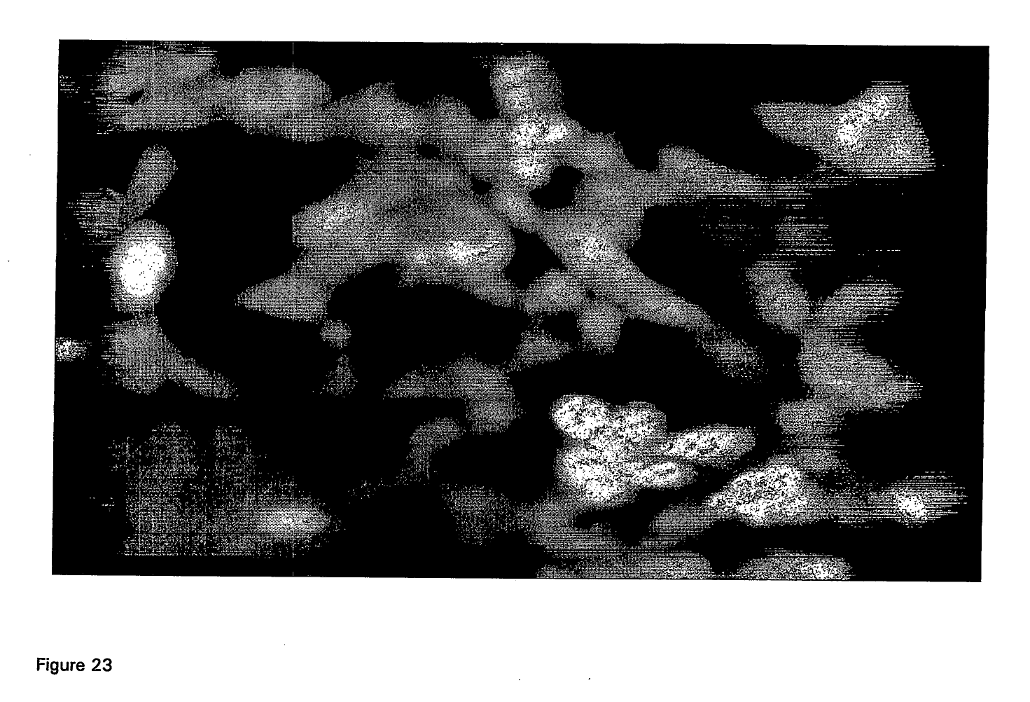

FIG. 23 shows HEK 293 cells: Delivery of lucifer yellow using poly-APS at 7° C.

### EXAMPLES

Sponge Toxin Preparations

Poly-APS (FIG. 1A) were purified from the marine sponge Reniera (=Haliclona) sarai Pulitzer-Finali (Haliclonidae). Aqueous extract containing poly-APS was passed through an ultrafiltration membrane (3 kDa cut off). Toxins were eluted from Sephadex G-50 and Sephacryl S-200 (Sep\check{c}ić et al 1997; J. Nat. Prod. 60 991-996). A halitoxin preparation was isolated from Callyspongia ridleyi. Butanol extraction containing halitoxins was applied to a lipophilic Sephadex LH-20 size exclusion column and the 5 to 6 kDa fraction isolated and studied (Jaspers et al 1994; J. Org. Chem. 59 3253-3255 and Scott et al 2000; J. Mem. Biol. 176 119-131). Stock solutions containing 5 mg/ml of either toxin preparation were diluted to produce poly-APS or halitoxin test solutions.

Cell Culture

Primary cultures of dorsal root ganglion (DRG) neurones were prepared following enzymatic and mechanical dissociation of dorsal root ganglia from decapitated 2-day old Sprague Dawley rats. The sensory neurones were plated on laminin-polyornithine coated coverslips and bathed in F14 culture medium (Imperial Laboratories) supplemented with 10% horse serum (Gibco), penicillin (5000 IU/ml), streptomycin (5000 mg/ml), NaHCO₃ (14 mM) and nerve growth factor (20 ng/ml). The cultures were maintained for up to 2 weeks at 37° C. in humidified air with 5% CO₂, and re-fed with fresh culture medium every 5-7 days.

HEK293 cells (human embryonic kidney cell line) were maintained in Dulbecco's modified Eagle's medium (DMEM). In preparation for transfection experiments cells were seeded 24 hours prior to experimentation at a density of 5×10⁵ cells per well in six well plates, the same seeding density was used for confocal and electrophysiology/calcium imaging experiments in 35 mm culture dishes.

Electrophysiology and Calcium Imaging.

All experiments were conducted at room temperature (approximately 23° C.). The whole-cell recording technique was used to study the actions of poly-APS and halitoxin preparations on membrane potential, input resistance (evaluated from 100-300 ms electrotonic potentials evoked by −30 to −200 pA current commands) and holding current. Whole cell recordings were made using an Axoclamp-2A switching voltage clamp amplifier operated at a sampling rate of 15-20 kHz. Low resistance (4-10 or 4-7 MΩ) borosilicate glass patch pipettes were fabricated using a Kopf model 730, needle/pipette puller. The neurones were bathed in a NaCl-based extracellular solution containing in mM: NaCl, 130; KCl, 3.0; CaCl₂, 2.0; MgCl₂, 0.6; NaHCO₃ 1.0, HEPES 10.0, glucose 5.0. NaCl-based extracellular solution with zinc was also made up separately and contained 1.5 mM zinc (zinc atomic absorption standard solution, Sigma). The pH and osmolarity of extracellular solutions were adjusted to 7.4 and 310-320 mOsmol/l with NaOH and sucrose respectively. The patch pipette solution contained in mM: KCl, 140; EGTA, 5; CaCl₂, 0.1; MgCl₂, 2.0; HEPES, 10.0; ATP, 2.0; and the pH and osmolarity were adjusted to 7.2 with Tris and 310-315 mOsmol/l with sucrose. For voltage clamp recordings, neurones were held at −90 mV and linear current-voltage relationships were generated with 100 ms voltage step commands to potentials between −170 and −60 mV. Poly-APS and halitoxin were applied to the extracellular environment by low pressure ejection via a blunt micropipette (tip diameter about 10 mm) positioned approximately 100 mm from the neurone being recorded. The cells were maintained in a bath and were not continually perfused, drug concentrations declined after pressure ejection as a result of diffusion.

The electrophysiological data were stored on digital audio tape (DAT) using a DTR-1200 DAT recorder (Biologic) and subsequently analysed using Cambridge Electronic Design voltage clamp software (version 6). For monitoring changes in membrane potential or holding current continuous records were obtained on a chart recorder (Gould 2200s pen recorder).

Cultured DRG neurones and HEK 293 cells were incubated for 1 hour in NaCl-based extracellular solution containing 10 μM fura-2AM (Sigma, 1 mM stock in dimethylformamide). The cells were then washed for 20 minutes to remove the extracellular fura-2AM and to allow cytoplasmic de-esterification of the Ca²⁺ sensitive fluorescent dye. The cells were constantly perfused (1-2 ml/min) and viewed under an inverted Olympus BX50W1 microscope with a KAI-1001 S/N 5B7890-4201 Olympus camera attached. The fluorescence ratiometric images from the data obtained at excitation wavelengths of 340 nm and 380 nm were viewed and analysed using OraCal pro, Merlin morphometry temporal mode (Life Sciences resources, version 1.20).

All data are given as mean±standard error of the mean (SEM) and statistical significance was determined, using the Student's two-tailed t test, paired or independent where appropriate and P values are reported in the text.

DNA Transfer

Plasmid cDNAs used were pEGFP-C1 (Clontech), an enhanced green fluorescent protein (EGFP) cDNA vector under control of a constitutively active SV40 promotor (pEGFP), human tumour necrosis factor receptor-2 (TNFR2) cDNA (provided by Werner Lesslauer, Basel, Switzerland) and pBABE hygromycin resistance cDNA (Stratagene). Control transfections were carried out using optimized lipofectamine (Invitrogen life technologies) lipid-micelle-mediated transfection protocol, which incubates cells with 1 μg cDNA and lipofectamine in the absence of serum for 3 h prior to re-introduction to serum-containing medium. A standard toxin transfection protocol was developed and optimised throughout the passage of this work. The protocol involved a 5 min serum-free cell incubation with 0.5 μg/ml of a sponge toxin preparation, followed by addition of 2.5 μg cDNA. After a further 3 hour incubation, DMEM medium was replaced with standard serum-containing medium. Lipofectamine and poly-APS stable transfections involved co-transfection with 1.0 μg or 2.5 μg cDNA respectively (either pEGFP or TNFR2 DNA) and 1.0 μg of pBABE cDNA. Colonies of stably transfected cells were selected in DMEM containing 100 μg/ml hygromycin B (Boehringer Mannheim). Once established, colonies were harvested using cloning discs and trypsin-EDTA and seeded into larger vessels until sufficient cells were available for analysis. In order to reduce variability cDNA was introduced into 1 ml preparations at a volume of 10 μl.

Crystal Violet Assay

Adherent cells were fixed in paraformaldehyde and stained in crystal violet dye (Sigma), subsequent elution and spectrophotometric analysis quantified the amount of intact viable cells following transfection.

EGFP Detection

Cellular expression of EGFP was evaluated using an Eppendorf fluorescence/visible light microscope set-up to directly assess the percentage of total cells fluorescing. Additionally, cells were imaged using a BioRad pradiance confocal system.

FACS Analysis

HEK293 cells stably over-expressing TNFR2 were dissociated from their culture vessels with 3 ml cell dissociation solution (trypsin free, Sigma) and assessed for TNFR1 and TNFR2 expression with specific mouse mAb (htr-9 and utr-1 respectively), secondary labelling with flouroisothiocyanate (FITC) anti-mouse IgG followed by Fluorescence-activated cell sorting analysis (Becton-Dickenson)

Confocal Analysis

HEK293-TNFR2 stable clones were plated into triplicate 35 mm dishes and fixed with ice-cold methanol. Individual dishes were probed with no antibody, TNFR1- or TNFR2-specific mouse mAb (htr-9 and utr-1 respectively) and then secondary labeled with FITC anti-mouse IgG. Cellular FITC labeling was then assessed using a BioRad μradiance confocal system, and was indicative of TNF receptor expression levels.

Temperature Studies

The actions of poly-APS on HEK 293 cells were evaluated at 12° C. HEK 293 cells were loaded (1 hour with fura2 AM (10 μM) in dimethylformamide) with the calcium sensitive dye and then continually perfused with NaCl-based extracellular medium at 12° C. Application of 0.1, 0.5 or 1.0 μM poly-APS evoked Ca²⁺ transients as measured by a change in fluorescence ratio. These data indicate that poly-APS-evoked pore formation can occur at 12° C. The results for 1.0 μM poly-APS are shown in FIG. 21.

Plasmid cDNA (pEGFP-C1 from Clontech), an enhanced green fluorescent protein (EGFP) cDNA vector under control of a constitutively active SV40 promoter was used. All transfection procedures were carried out as previously optimised using poly-APS except the temperature was reduced to 12° C. The protocol involved a 5 minute serum-free cell incubation with 1.0 μg/ml of a sponge toxin preparation, followed by addition of 2.5 μg cDNA, after a further 3 hours incubation serum was added and cells were maintained in culture at 37° C. Transfection was evaluated after 24 hours. The results are shown in FIG. 22. In some preparations 30% transfection efficiency was achieved.

HEK 293 cells were exposed to 0.5 μg/ml poly-APS for 5 minutes and then poly-APS & 1 mM lucifer yellow for 3 hours. Washing (NaCl-based saline), pre-incubation in toxin and incubation in lucifer yellow were carried at 7® C. Lucifer yellow was loaded into almost all cells when the experiment was conducted at 7® C. The intracellular level of lucifer yellow was reduced but was still apparent after returning the cells to culture medium and incubating at 37® C for 24 hours. In the absence of poly-APS, no lucifer yellow was found intracellularly. The results are shown in FIG. 23.

These temperature experiments suggest that internalisation of the macromolecules is not occurring by endocytosis because the process of endocytosis is temperature dependent and is blocked below 12® C.

Delivery of the Cytoskeletal Protein Tau and Poly-APS into the Rat Hippocampus

This was performed using well known methods of the art. The tau protein plus poly-APS, as appropriate, were injected into the hippocampus through a bore hole. The concentration of poly-APS used was between 0.005 μg/ml and 5 μg/ml, preferably 0.005 μg/ml or 5 μg/ml. The concentration of tau was 1 μg/ml. Okadaic acid was administered via a mini pump at a concentration of 1.2 nM for 7 days.

Obtaining Ex Vivo Hippocampal Slices.

Recordings

Hippocampal slices were obtained from pigmented Lister-Hooded rats 3-4 weeks after birth. Methods have been described in detail previously (White and Platt (2000) Eur J Neurosci 12 3155-3162). Briefly, terminally anaesthetised animals were decapitated, the brain quickly removed and placed into ice-cold artificial cerebrospinal fluid (aCSF, See Table 3 for composition) saturated with 95% 0₂/5% CO₂, pH 7.4. A vibrotome (Oxford Instruments, UK) was used to prepare 350-μm parasagittal hippocampal slices, which were subsequently maintained in aCSF at ca 30° C. for at least an hour before recordings commenced.

Measurement of Long-Term Potentiation

For extracellular field recordings, slices were submerged in a recording chamber; a monopolar stimulation electrode (ca 0.5 MΩ, WPI, USA) driven by a DS2 A stimulus isolator (Digitimer, UK) was placed in the outermost part of the optic layer and a recording glass electrode (Clark, UK) filled with aCSF was placed in the superficial grey layer adjacent the stimulation electrode. Evoked glutamate-mediated fEPSPs were recorded (Axoclamp 2A, bridge mode; Axon, USA), stored on a computer and analysed (amplitude and slope of the initial rising phase, from fibre volley to peak; PWIN software package). Activity was also continuously monitored on an oscilloscope (Tektronix, USA). As in previous papers (Platt and Withington (1998) Neuropharmacology 37 1111-1122; White and Platt (2000) Eur J Neurosci 12 3155-3162), slices were stimulated every 30 s at 70-80% of the maximum fEPSPs until a stable baseline was obtained for at least 20 min.

Drugs

LTP_{G} was induced by superfusion of 3 mM GABA for 7 min and was washed out with aCSF for another 60 min. In subsequent experiments the perfusion solution was manipulated by either decreasing potassium ions, calcium ions, or by replacing bicarbonate buffer with N-[hydroxyethyl]piperazine-N-[2-ethanesulfonic acid] (HEPES; see Table 3 for details of aCSF composition). The pH was monitored repetitively, in particular for experiments in HEPES-buffered solution, and found to be stable throughout the experiments. A CA inhibitor ethoxyzolamide (EZA; 50 μM) was also used. NKCCl and KCC2 inhibitors bumetanide (50 μM) and furosemide (100 μM) were also tested. All drugs were purchased from Sigma Aldrich, USA apart from the salts used for aCSF which were obtained from BDH, UK.

TABLE 3
Composition of extracellular solutions
(artificial cerebrospinal fluid) in Mm

| | | Low | Low | |
|---|---|---|---|---|
| Compound | Control | K+ | Ca²⁺ | HEPES |
| NaCl | 127 | 129.5 | 130 | 127 |
| KCl | 4 | 1.5 | 4 | 4 |
| CaCl₂ | 2.5 | 2.5 | 1 | 2.5 |
| KH₂PO₄ | 1.5 | 1.5 | 1.5 | 1.5 |
| MgCl₂ | — | — | — | — |
| MgSO₄•7H₂O | 1.3 | 1.3 | 3 | 1.3 |
| Glucose | 10 | 10 | 10 | 10 |
| NaHCO₃ | 25 | 25 | 25 | — |
| HEPES | — | — | — | 10 |

Data Analysis

LTP_{G} was calculated relative to baseline values in percent for both slop and amplitude. Statistical significance was assessed for data before and 1 h after GABA application (three consecutive values were averaged in each case) using Prism for Windows (Version 3.00; GraphPad Software, USA). One-way ANOVA with Dunnett's multiple comparison post hoc test was performed for LTP_{G} comparison 60 min after GABA application. Unpaired and paired two-tailed Student's t-tests were also used to compare LTP_{G} in different conditions and to compare baseline and LTP_{G} values within groups, respectively.

The Open-Field Water Maze For Rats

Apparatus

The open-field water maze used has been described previously. In brief, a pool 2 m in diameter was filled with water at a temperature of 25±2° C., to a depth of approximately 50 cm, and rendered opaque by the addition of latex subflooring compound. A platform, 11 cm in diameter, submerged approximately 2 cm below the water surface, provided the only means of escape for the rats. The pool was located in a room that provided plenty of extramaze cues. A curtain could be drawn around the pool's circumference.

Behavioural Paradigm

During habituation and all subsequent training and testing, the following conditions applied. Four cardinal points (north, south, east and west) were semirandomly chosen as start locations, and rats were released facing the wall of the pool. If the rats could not locate a platform within 120 s, the trial was terminated and the rat was guided to the platform by the experimenter. After having found the platform, rats were left on it for 30 s before being placed back in their cage. Heating lamps placed in a room adjacent the water maze room were used to dry the rats after testing.

Results

Irreversible and Reversible Actions of Poly-APS on Electrophysiological Properties of Cultured DRG Neurones

Extracellular application of 50 μg/ml poly-APS for approximately 20 seconds resulted in an irreversible collapse in membrane potential and a dramatic fall in input resistance in all DRG neurones studied (FIG. 1B). The mean membrane potential was depolarised from −65±1 mV to −9±3 mV (n=17; P<0.0001) and this was associated with a reduction in input resistance from 350±79Ω to 56±27Ω (n=17; P<0.0001). No recovery was seen 120 minutes after removal of the perfusion pipette containing Poly-APS. At concentrations of 5 and 0.5 μg/ml, poly-APS evoked reversible depolarisations and associated decreases in input resistance (FIG. 1C). Poly-APS showed a degree of dose dependent action when the sponge toxins were applied for approximately 20 seconds, the mean percentage reductions in input resistance were 84%, 67% and 17% for 50, 5, and 0.5 μg/ml poly-APS respectively. Recovery periods varied but were seen within 20 minutes of removing the perfusion pipette containing poly-APS. In responses to 5 μg/ml poly-APS, 6 out of 8 neurones showed at least partial recovery of membrane potential and input resistance after toxin application (FIG. 2A and FIG. 2B).

Reversible Actions of Poly-APS on Cell Membrane Calcium Permeability

Consistent with the electrophysiological results, Ca²⁺ imaging experiments on cultured DRG neurones showed that poly-APS evoked irreversible (FIG. 3B) and reversible (FIG. 3B) increases in intracellular Ca²⁺. Although the responses were very variable a dose-dependent trend was clearly apparent. Poly-APS (5 μg/ml) produced mainly responses that either only partially recovered or were irreversible and 0.05 μg/ml poly-APS evoked predominantly transient responses with 4 out of 45 neurones failing to respond (Table 1). These results highlight the narrow concentration range required for reversible pore formation and the reason for cell death at high concentrations of poly-APS.

TABLE 1
Reversible and irreversible rises in intracellular
Ca²⁺-evoked by poly-APS as detected using the
Ca²⁺-sensitive ratiometric dye fura-2.
Reversible is defined as a transient event that showed recovery
of 75% or more. Irreversible is defined as a sustained event
that only decayed by 10% or less during washing.

| | Irreversible | Amplitude | | |
|---|---|---|---|---|
| Poly-APS | (I) or | of response | No. of | % of |
| Concentration | Reversible | in ratio | DRG | DRG |
| (mg/ml) | (R) responses | units ± S.E.M. | neurones | neurones |
| 5* | I | 2.54 ± 0.28 | 15 | 29% |
| 5* | R | 2.18 ± 0.45 | 9 | 17% |
| 0.5 | I | 3.16 | 1 | 3% |
| 0.5 | R | 2.58 ± 0.51 | 31 | 97% |
| 0.05 | I | — | 0 | 0% |
| 0.05 | R | 2.53 ± 0.4 | 41 | 100% |

*28 neurones (54%) showed partial recovery and were not included in this analysis.

No significant differences were observed when comparing the mean amplitudes of irreversible and reversible rises in intracellular Ca²⁺ and the mean amplitude of events evoked by 5, 0.5 and 0.05 μg/ml poly-APS. This relates partially to the considerable variability of responses, for example the maximum and minimum changes in fluorescence ratio produced by 5 μg/ml poly-APS were 5.42 and 0.48, and even in neighbouring neurones during the same experiment great differences in responses were seen. Additionally, poly-APS was applied for 2 to 2.5 minutes, which was significantly longer than the periods the toxins were applied for in the electrophysiological experiments. One benefit of using cultured DRG neurones in this study was that these neurones express voltage-activated Ca²⁺ channels, which can be used to assess the viability of neurones after exposure to poly-APS. The experimental trace in FIG. 3C shows that Ca²⁺ transients evoked by a depolarising pulse of extracellular solution containing KCl (30 mM) could be produced before and after application of 0.05 μg/ml poly-APS. Accordingly, it was shown that repeatable poration could be obtained and entry of Ca²⁺ via voltage-gated channels remained intact after application of low doses of poly-APS indicating that at least at low concentrations cytotoxic damage did not occur as a result of pore formation. It was however surprising that voltage-gated Ca²⁺ channels still functioned after a period of poration given the sensitivity of Ca²⁺ channels to intracellular Ca²⁺-induced inactivation.
Action of Poly-APS and Halitoxin Preparations on Membrane Potential, Input Resistance and Ca²⁺ Permeability of HEK293 Cells

The actions of poly-APS and the halitoxin preparations on membrane potential, input resistance and Ca²⁺ permeability of HEK293 cells were also evaluated. A 20 second application of halitoxin (0.5 and 5.0 μg/ml) predominantly resulted in irreversible effects on the electrophysiological properties of HEK293 cells (FIG. 4B). Halitoxin (0.5 μg/ml) reduced membrane potential from −45±5 mV to −16±5 mV (n=6; P<0.003) and partial but significant recovery to −26±4 mV (n=6; P<0.02) was observed 10-20 min after toxin application. Halitoxin (5 μg/ml) reduced membrane potential from −46±3 mV to −3±1 mV (n=5; P<0.003) with no significant recovery. Similar trends were observed when input resistance was determined from the electrotonic potentials evoked by hyperpolarising current commands. For example the resting input resistance was reduced from 903±313 M to 315±206 MW (n=6; P<0.04) by 0.5 μg/ml halitoxin but no significant partial recovery was observed. There was also evidence that the effects on membrane potential were apparently dose dependent with 0.5 and 5 μg/ml halitoxin reducing membrane potentials by 64±10% (n=6) and 94±3% (n=5; P<0.03) respectively. In contrast the actions of poly-APS applied for 20 seconds were predominantly reversible both at 5 μg/ml (n=6 out of 9 cells, FIG. 4C and FIG. 4D) and at 0.5 μg/ml (n=6 out of 9 cells). The apparent dose dependency was less apparent and both 5 and 0.5 μg/ml poly-APS gave equivalent electrophysiological responses to those obtained with 0.5 μg/ml halitoxin.

Experiments using the ratiometric dye fura-2 also indicated that both halitoxin and poly-APS evoked large changes in intracellular Ca²⁺. Initially a protocol was used to examine the dose-dependent effects of the sponge toxins. Halitoxin or poly-APS were applied for 140 seconds at doses of 0.005, 0.05, 0.5 and 5.0 μg/ml with variable wash periods between each application. Few cells responded to the sponge toxins when they were applied for 140 seconds at doses of 0.005 μg/ml (10 of 115 cells for Halitoxin and 2 of 90 cells for poly-APS). Accordingly, 0.005 μg/ml appears to be the threshold concentration for a detectable Ca²⁺ transient in HEK293 cells. The halitoxin preparation produced some anomalous results because cell lysis and dye loss were usually observed with 5 μg/ml when the cells had been exposed previously to the three lower doses of toxin. The mean peak changes in fluorescence ratios observed were 0.2±0.03 (n=10), 0.43±0.03 (n=80) and 1.09±0.04 (n=114) with 0.005, 0.05 and 0.5 μg/ml halitoxin respectively. However, at least in some cells, transient changes in intracellular Ca²⁺ were observed with 0.05 and 0.5 μg/ml halitoxin (FIG. 5A). Dose-dependent and predominantly reversible responses were obtained with poly-APS (0.005-5 μg/ml; FIG. 5B). The mean peak changes in fluorescence ratios observed were 0.1 (n=2), 0.65±0.06 (n=63), 0.88±0.06 (n=83) and 1.17±0.05 (n=89) with 0.005, 0.05, 0.5 and 5.0 μg/ml poly-APS respectively. The different n numbers for both sets of data are indicative of varied threshold sensitivities of different HEK293 cells to the sponge toxins. In this connection, some cells did not respond to 0.05 μg/ml toxin. Separately applying single doses of each toxin clearly showed the different amplitudes and natures of responses produced by the sponge toxins (FIG. 5C and FIG. 5D). All cells studied responded to 0.5 and 5.0 μg/ml toxins and, therefore, these doses were used in subsequent experiments.

Actions of Zinc on Pore Formation by Poly-APS.

The hemolytic actions of poly-APS have been found to be attenuated by zinc, most likely in the ionic form Zn²⁺, so the protective properties of zinc (Zn²⁺) when applied continually or after pore formation by poly-APS was investigated. Experiments were carried out on the same cultures of DRG neurones to allow comparisons between results obtained using the two protocols. Using the first experimental protocol, poly-APS (50 μg/ml) reduced the membrane potential from −66±2 mV to −13±6 mV (P<0.0001) but during subsequent application of extracellular solution containing 1.5 mM zinc the membrane potential did not significantly recover and had a value of −17±6 mV (n=8). A similar pattern of results was obtained for input resistance measurements which had values of 252±52Ω, 13±3Ω (significance compared to control P<0.005) and 19±7Ω (n=8), under control conditions, during application of 50 μg/ml poly-APS and during application of zinc. It was clear from this experiment that once poly-APS had produced pores or lesions in the cell membrane zinc failed to attenuate the conductances (FIG. 6A). However, using the second experimental protocol which involved continually bathing neurones with extracellular solution containing 1.5 mM zinc resulted in significant inhibition of poly-APS actions (FIG. 6B and FIG. 6C). In the presence of zinc the resting membrane potential was −47±7 mV and application of poly-APS (50 μg/ml) with zinc significantly reduced the membrane potential to −27±6 mV (n=7; P<0.07). Similarly in the presence of zinc the input resistance was 261±34Ω and application of poly-APS (50 μg/ml) with zinc significantly reduced the input resistance to 114±62Ω (n=7; P<0.05). FIG. 6C shows a bar chart of data normalised with respect to the resting values to illustrate the significant attenuation of poly-APS effects when zinc was present prior to and during application of the toxin.

Experiments were then conducted under voltage clamp conditions to remove the influence of voltage-activated channels. Neurones were held at −90 mV and currents were activated by poly-APS (10 μg/ml) in the presence and absence of 1.5 mM zinc containing solution. Current voltage relationships were generated between −160 mV and −70 mV and subtracted to produce difference current voltage relationships for the poly-APS evoked currents (FIG. 7B). The results with 10 μg/ml poly-APS showed that although zinc significantly reduced the action of the toxin its effects were not abolished so the concentration of poly-APS was reduced. The presence of 1.5 mM zinc containing solution prevented significant activation of inward currents evoked by 0.5 μg/ml poly-APS. In the absence of zinc the holding current required to clamp the cell membrane at −90 mV increased from −150±30 pA to −600±190 pA (n=5; P<0.04) during application of 0.5 μg/ml poly-APS. In contrast with 1.5 mM zinc containing solution continually present, the holding current was not significantly increased by poly-APS (0.5 μg/ml) from a resting level −120±30 pA to −170±70 pA (n=6; NS). The mean amplitudes of currents evoked by poly-APS in the presence and absence of zinc were also significantly different (P<0.05; FIG. 7B).

Imaging experiments were then carried out to determine whether zinc would attenuate the rise in Ca²⁺ evoked by poly-APS. Protocols were again designed to assess the ability of zinc to inhibit rises in Ca²⁺ produced by poly-APS, when applied before or after poration of the cell membrane. It was not possible to clearly characterise the effects of zinc when applied after poly-APS because of the slow partial recovery of the responses that occurred during washing (FIG. 8A). Application of zinc itself produced a modest but significant increase in fluorescence ratios above basal levels, and poly-APS (5 μg/ml) applied with zinc subsequently evoked a sustained rise in fluorescence ratio (FIG. 8B). However, these responses in the presence of zinc were significantly smaller than the responses evoked by poly-APS in the absence of zinc (FIGS. 8A, 8B & 8C). The change in the mean fluorescence ratios evoked by poly-APS in the absence and presence of zinc were significantly different and had values of 3.46±0.18 (n=46) and 1.2±0.05 (n=13; P<0.0001) respectively. These experiments were conducted on two populations of neurones; however, it was hoped that by lowering the poly-APS concentration it would be possible to record poly-APS responses in the absence and presence of zinc in the same neurones. Preliminary observations showed that repeatable responses to poly-APS could be obtained from some neurones. In these experiments the mean control fluorescence ratio was 0.9±0.03. The first and second applications of poly-APS (0.5 μg/ml) resulted in significant increases in fluorescence ratio to values of 5.36±1.38 and 3.45±0.55 (n=10; P<0.01 and 0.05 compared to control). The first and second responses to poly-APS were not significantly different.

Subsequently experiments were conducted with 0.05 μg/ml poly-APS. For this experiment the mean fluorescence ratio under control conditions was 0.94±0.02. The first response to poly-APS (0.05 μg/ml) resulted in a significant rise in fluorescence ratio to 2.53±0.31 (n=22; P<0.01); subsequent application of zinc and a second application of poly-APS (0.05 μg/ml) produced an attenuated response as the fluorescence ratio increased to 1.85±0.16 (n=22). The second response in the presence of zinc was significantly different from both the basal levels (P<0.01) and the first response (P<0.05). The second response is likely to be an overestimation of the poly-APS actions because zinc itself produced more pronounced effects than predicted (FIG. 9A). Similar responses were obtained when poly-APS and zinc were simultaneously applied to produce the second response (FIG. 9B). It is likely that zinc passed through residual pores in the membrane produced during the first application of poly-APS and resulted in a slow sustained increase in fluorescence ratio.

Actions of Zinc on Pore Formation by Poly-APS in HEK 293 Cells.

One difficulty of assessing rises in intracellular Ca²⁺ evoked by poly-APS in cultured DRG neurones is that in addition to Ca²⁺ entry through pores formed by poly-APS, rises in intracellular Ca²⁺ will also be produced by activation of voltage-gated Ca²⁺ channels and mobilisation of Ca²⁺ from stores. Although it should be noted that repeatable responses to low doses of poly-APS were obtained, it is possible to underestimate or overestimate the inhibitory effects of zinc given the other pathways for raising intracellular Ca²⁺. For this reason preliminary experiments were conducted on HEK 293 cells which did not express voltage-gated Ca²⁺ channels. As with the DRG neurones, poly-APS (1.5 μg/ml) evoked repeatable rises in intracellular Ca²⁺. The mean fluorescence ratios for the first and second applications of poly-APS were 2.31±0.25 and 1.96±0.3 (n=7; not significantly different).

Experiments were then carried out to assess the influence of zinc on the Ca²⁺ entry produced by poly-APS. Under control conditions the mean fluorescence ratio was 1.16±0.07 and brief application of extracellular recording solution containing 30 mM KCl caused no significant response (n=11; FIGS. 10A & 10B). Application of poly-APS (0.5 μg/ml) in the absence and presence of zinc evoked responses, and it was clear from these results that zinc attenuated the rise in intracellular Ca²⁺ produced by poly-APS.

Thus, it was clearly demonstrated that zinc (Zn²⁺), if continually present during application of poly-APS, could attentuate the effects of the sponge toxin preparation on membrane potential, input resistance, whole cell currents and Ca²⁺ permeability.

Further, the results show that poly-APS can reversibly form pores in the membranes of DRG neurones and HEK 293 cells, and that applying extracellular zinc with the preparation of sponge toxins can attenuate these actions. The survival of cells even after dramatic but temporary changes in membrane potential and input conductances means that transient pore formation by low doses of poly-APS could be used to deliver materials to the intracellular environment, without cell damage.

Previous studies have been performed on pore properties and potential mechanisms of passive DNA flow. Furthermore single stranded polynucleotide RNA and DNA molecules have been shown to traverse lipid bilayers treated with Staphylococcus aureus α-toxin or α-hemolysin, which creates membrane pores of 1-2.5 nm diameter. The passage of these nucleotide molecules across the bilayer was signalled by fluctuations in channel conductances as individual molecules passed through, and allowed molecular characterisation. A number of other diverse molecules have been similarly shown to create perforations in lipid bilayers, including perforin, complement, detergents such as α-escin and saponin, and alamethicin but as yet none has been shown to provide passage for DNA. More conventional approaches to DNA transfer across lipid bilayers and into intact cells include lipid-micelle-mediated transfection, e.g. lipofectamine, electroporation and micro-injection, but these approaches yield variable results and can be difficult to control, often requiring specialist equipment or laborious pretreatments and optimisation.

Poly-APS has distinct but comparable properties with cationic surfactants but variation in the extent of polymerisation of the poly-APS appears to modulate the biological activity in an unpredictable manner. In the present invention we have identified reversible effects of poly-APS on cell membrane properties. Previously, the related but lower molecular weight preparation of halitoxins evoked irreversible pore formation and associated changes in cell membrane input conductance. There are several possible explanations for the reversible changes in membrane potential, input resistance, currents and intracellular Ca²⁺ seen with poly-APS. Firstly, the larger degree of polymerisation in poly-APS, relative to smaller molecules composed of fewer monomeric units, may result in less stable interactions in the cell membranes and allow wash out of the larger pore forming sponge toxins with a resulting temporary pore formation. Secondly, and perhaps more likely is the possibility that the larger poly-APS may be sufficiently flexible so that lipids can re-arrange themselves after pore formation and thus block the ion conducting pathways through the cell membrane. This may occur due to “hydrophobic collapse” when the toxin is in the membrane. In such a circumstance, alkyl chains may surround all the pyridinium groups so that they are compatible with the membrane. The ability of poly-APS to form non-covalently bound aggregates with a mean hydrodynamic radius of 23 nm and its cationic charge density and hydrophobicity may be the key factors in transient pore formation. Repeatable poration could be obtained, and entry of Ca²⁺ via voltage-gated channels remains intact after application of low doses of poly-APS which indicates that at least at low concentrations cytotoxic damage does not occur as a result of pore formation.

Poly-APS produced highly variable Ca²⁺ transients, which could reflect the nature of Ca²⁺ stores within DRG neurones and variable expression of voltage-activated Ca²⁺ channels. Poly-APS evoked membrane potential depolarisation would activate endogenous Ca²⁺ channels and both entry of Ca²⁺ through poly-APS pores and native channels would mobilise Ca²⁺ from stores through Ca²⁺-induced Ca²⁺ release. Variability in responses to KCl-evoked depolarisation has been reported in DRG neurones but single neurones can respond consistently to repeated stimulation with KCl and poly-APS. Crude organic extracts containing bioactive pyridinium alkaloids from the sponge Amphimedon viridis show selective antibacterial activity. Some marine, bacterial strains that possibly have symbiotic relations with the reef sponges were resistant to the toxin extracts. Poly-APS has not been found to have antibacterial activity against terrestrial and pathogenic gram-positive and gram-negative bacteria. Little comparative data has been obtained on the actions of sponge toxins on diverse pro- and eucaryotic cell types. However, our findings raise the possibility that variable sensitivity of cells and organisms to poly-APS is based on their intrinsic membrane composition and properties. The variability in the responses to poly-APS seen in the DRG neurone cultures could in part reflect the heterogeneous population of neurones in this preparation, with sensory neurones varying in their anatomical, biophysical and pharmacological characteristics.

It was also clearly demonstrated above that zinc, if continually present during application of poly-APS, could attenuate the effects of the sponge toxin preparation on membrane potential, input resistance, whole cell currents and Ca²⁺ permeability. This could involve an interaction between zinc and the cell membrane to prevent access of the toxin to sites on the membrane. The Shai-Matsuzaki-Huang model for actions of antimicrobial peptides may provide a mechanism for pore formation by poly-APS. The initial stage in the model involves pore-forming molecules carpeting the outer membrane leaflet and this may be prevented by extracellular Zn²⁺ binding to negative charges on the membrane. The affinity of zinc could be much greater than that of pyridinium compounds and hence as seen in this study premixed poly-APS with zinc results in reduced poration. Thus zinc might prevent the further stages in pore formation, which are integration of the pore former into the membrane, thinning of the outer leaflet, phase transition and “wormhole” formation with the poly-APS molecules finally spanning the membrane producing a conductance pathway. Alternatively, zinc may interact with poly-APS directly to disrupt its biological activity. Previous work on erythrocytes has suggested that Zn²⁺ and other divalent cations (Hg²⁺) close pores produced by poly-APS but not lysis caused by hypoosmotic shock. This has not been demonstrated in this study, although, as suggested above, the interactions of poly-APS with membranes may vary and thus influence the protective actions of zinc, and the mechanism of erythrocyte lysis is made distinct by the osmotic influence of haemoglobin.

Fura-2 was used to measure changes in intracellular Ca²⁺ produced following pore formation by poly-APS, and significant reductions in fluorescence ratios were seen when zinc was applied. However, several features of the Ca²⁺ imaging experiments require further consideration. Firstly, fura-2 has also proved useful in measuring small increases in intracellular zinc because of its high zinc sensitivity (K_{D} values ˜2 nM), which is about 100 times lower than that for Ca²⁺. The small sustained responses consistently observed with zinc present seem to reflect fura-2 detecting zinc rather than Ca²⁺, the sustained nature of these events indicates that unlike Ca²⁺, zinc was not readily removed from the intracellular compartment. This suggests that zinc can pass through the poly-APS-evoked pores but that Ca²⁺ entry was attenuated to a greater extent than the data indicates. Combined with the electrophysiological data, the imaging studies with fura-2 indicate that zinc reduced levels of poration, and may also inhibit Ca²⁺ entry through the pores that are formed by poly-APS. However, it is possible that zinc interactions with fura-2 compete significantly to reduce Ca²⁺ detection. Secondly, zinc has been shown to inhibit voltage-activated neuronal conductances, including those carried by Ca²⁺ which may contribute to the poly-APS responses, and this could result in an overestimation of the inhibitory action of zinc on poly-APS effects. However, data has also been obtained from HEK 293 cells and under voltage clamp that are consistent with zinc inhibiting poly-APS-evoked pore formation independently of voltage-activated Ca²⁺ channels.

Poly-APS can reversibly form pores in the membranes of DRG neurones and HEK 293 cells, and applying extracellular zinc with the preparation of sponge toxins can attenuate these actions. The survival of cells even after dramatic but temporary changes in membrane potential and input conductances suggests that transient pore formation by low doses of poly-APS can be used to deliver materials to the intracellular environment, without cell damage.

The transient, reversible nature of the pores formed by the sponge toxins prompted further study to investigate whether these transient membrane pores/lesions were sufficient to conduct plasmid cDNA across the lipid bilayer.

In this connection, qualitative assessment of HEK293 cells incubated with 0.5 μg/ml poly-APS or halitoxin and 2.5 μg pEGFP indicated successful transfection, as a number of fluorescent cells were observed (FIG. 11A, lower panels). This was in contrast to cells incubated with toxin alone (FIG. 11A upper panels) or pEGFP alone, which were not transfected and displayed no fluorescent cells. Use of a commercial transfection reagent (lipofectamine) to transfect HEK293 cells (FIG. 11A, left-hand panels) provided a positive control for both the PEGFP cDNA and the imaging of transfected fluorescent cells. Extensive optimisation experiments were required to maximise transfection of HEK293 cells using sponge toxins and to provide a standardised protocol. Such experiments investigated the influence on transfection efficiency of a number of variable factors and assessed both the percentage of cells transfected and cell survival.

A range of cDNA concentration experiments indicated that 2.5 μg pEGFP maximises transfection efficiency without inducing intolerable toxicity. The use of lesser quantities of cDNA, whilst being far less toxic, displayed low transfection efficiency, with greater quantities producing the opposite effect (high toxicity, moderate transfection efficiency).

Experiments focussing on the duration of incubation with toxin/cDNA revealed that a 5 min incubation with toxin prior to cDNA addition produced higher levels of transfection compared to simultaneous addition or longer periods of toxin pre-incubation. Furthermore, a period of 180 min incubation was optimal following addition of cDNA, with shorter periods reducing transfection efficiency and longer periods proving too toxic. Serum has a profound effect on lipid-mediated cDNA transfection, and so it was investigated whether similar effects would be seen with toxin-mediated transfection. Serum-free conditions proved most effective for toxin-mediated transfection, without raising toxicity levels, and in addition a return to standard 10% serum-containing medium following transfection was more beneficial than raising serum levels to 20% or above. Quantification of PEGFP transfection indicated that 0.5 μg/ml poly-APS/halitoxin achieved higher levels of transfection than either 0.05 μg/ml or 5.0 μg/ml (FIG. 11B). Comparable levels of transfection were achieved with both toxins, although at 0.5 μg/ml, poly-APS achieves marginally higher transfection success (FIG. 11B). These concentration-driven toxin activities coincide with the electrophysiological toxin profiles presented in FIGS. 4 and 5. Although these transfection levels are 4-fold lower than lipofectamine transfection efficiency, they are raised considerably above cDNA-only control levels (consistently 0). Analysis of the lethality of these toxins suggests that at both 0.05 μg/ml and 0.5 μg/ml neither of the toxins induce cell death above the tolerable level of death seen in lipofectamine-mediated transfection (typically 88-95% survival of HEK293 cells) (FIG. 11C). However, approximately 10-12 fold more death occurred with 5.0 μg/ml poly-APS and halitoxin (12% and 6% survival respectively) (FIG. 11C), suggesting a dose-dependent sponge toxin toxicity, with cells tolerant up to 0.5 μg/ml toxin. Furthermore, this very high toxicity explains the low proportion of fluorescent cells (FIG. 11B), with the toxin killing the cells as opposed to transfecting them at 5.0 μg/ml. This compliments data presented in FIGS. 5A and 5B concerning dose-dependent reduction in pore reversibility, which may be a factor contributing heavily to toxicity.

Stable transfection of HEK293 cells using 0.5 μg/ml poly-APS further illustrates this toxin as a potentially useful transfection tool. Following selection of stable colonies in hygromycin B selection reagent, a large number of colonies remained in 6-well plates transfected using lipofectamine or poly-APS, in contrast to control cells which had no hygromycin B resistant colonies (Table 2), indicative of hygromycin B lethality to normal cells.

TABLE 2
Comparison of lipofectamine and poly-APS transfected stable
colony number. Colonies of hygromycin B selected HEK293 cells
stably transfected with pEGFP or TNFR2 cDNAs using poly-APS
or lipofectamine were counted in culture wells prior to harvesting.
Upper panel shows comparison of lipofectamine and poly-APS colony
numbers, n = 5 and n = 10 respectively. Lower panel
split poly-APS stable data into either pEGFP or TNFR2 expressing
colonies, n = 5 in both cases.

| | Mean colony Number |
|---|---|
| Vehicle | |
| None | 0 |
| Lipofectamine | 18.5 ± 4.5 |
| poly-APS | 7.1 ± 1.4 |
| Poly-APS stable clones cDNA | |
| pEGFP | 6.2 ± 2.0 |
| TNFR2 | 8.0 ± 2.0 |

In addition, although lipofectamine is more effective than poly-APS at stably transfecting HEK293 cells (2.5-fold more colonies in lipofectamine plates), a lower yet still relevant level of transfection is achieved using poly-APS than commercial transfection reagents (Table 2 and FIG. 11B). Moreover, confocal visualisation of pEGFP stable clones suggests larger numbers of positive cells in lipofectamine wells, compared to poly-APS transfected wells, although again poly-APS shows considerably larger numbers of stable cells compared to controls (FIG. 12A). The type of cDNA being co-introduced to the cell along with pBABE has no bearing on the success of stable expression, with poly-APS-mediated stable transfection of PEGFP and TNFR2 cDNA producing comparable numbers of stable colonies. Analysis and comparison of TNF-receptor levels in HEK293 cells, and two TNFR2 stably transfected clones, show relatively low and comparable labelling of TNFR1 in all three cell types (FIG. 12B), consistent with wild-type TNFR1 expression. In contrast, only clone 4 shows raised TNFR2 labelling, indicating successful stable transfection of TNFR2 cDNA. Clone 7 does not show an enhanced TNFR2 expression level, with very low labelling similar to control cells (FIG. 12B), suggesting only pBABE and not TNFR2 cDNA has been successfully transfected, resulting in expression of only the hygromycin resistant phenotype. Study of the same cells using FACS shows a similar rightward shift in fluorescence intensity for all three cell types when labelled with TNFR1-specific antibodies compared to secondary antibody alone (FIG. 12C), indicating similar levels of TNFR1 in the three cell types. However no TNFR2 shift is visible in untransfected or clone 7 cells with TNFR2 histogram lines overlying secondary antibody alone lines (FIG. 12C), suggesting little or no TNFR2 expression in the Hygromycin-resistance-only stable clone 7, as is seen in wild-type HEK293 cells. In higher expressing stable clone 4, a considerable increase in fluorescence intensity is apparent in TNFR2 labelling, consistent with enhanced TNFR2 expression (FIG. 12C). Thus, clone 4 stably co-expresses both hygromycin resistant and TNFR2 enhanced phenotypes, following initial transfection with poly-APS, as proved by both confocal and FACS analysis, demonstrating the usefulness of these reversible toxins as transfection reagents. There have been reports of the ability of keratinocytes to incorporate plasmid DNA without the use of transfection reagents. However, this natural DNA uptake process seems to be particular to the keratinocyte cell phenotype with surrounding fibroblasts unable to naturally incorporate foreign genetic information. The existence of plasmid DNA-binding proteins specific to keratinocytes was purported; however, it is clear that such mechanisms do not exist in HEK293 cells, as cDNA incorporation was not detected in the absence of transfecting reagent (FIGS. 11A and 12A and Table 2).

The present invention provides that a simple incubation protocol using poly-APS can enable the introduction of large pieces of super-coiled DNA into living cells without irreparable cell damage. This novel and reproducible approach allows both transient and stable transfection conveniently with plasmid DNA, which has never before been achieved with pore forming molecules of this nature.

Consistent with findings in cultured dorsal root ganglion neurones and F-11 cells, halitoxin and poly-APS preparations produced conductance increases and raised intracellular Ca²⁺ in a manner consistent with pore or lesion formation in the cell membranes of HEK293 cells. It seems likely that the degree of polymerisation influences how readily the responses reverse; however, both sponge toxin preparations (halitoxin and poly-APS) contain two or more distinct alkyl pyridinium compounds. In addition to size of alkyl pyridinium compounds, issues of aggregation and lipid membrane constituents are likely influences on pore formation and cell recovery, and even between cells from a cell line considerable variability in responses was observed.

The exact pore size formed by poly-APS and halitoxin preparations to allow transport of plasmid cDNA inside cells is not presently known, although it has been predicted to be 2.9 nm. It is known that the pore size must be greater than 2.6 nm as the maximal Staphylococcus aureus α-toxin pore size is only sufficient to allow flux of ions and short (<200 bp) single-stranded oligonucleotides but not other biomolecules such as double-stranded DNA or protein. Moreover, the ionic components of Staphylococcus aureus α-toxin hinder DNA movement through the pore of its multiple subunit form. Although only marginally larger in diameter, the estimated poly-APS aggregate pore does not contain a highly charged regions that would repel DNA and preclude its ability to allow plasmid DNA transfer into cells. Clearly the pore size must be greater than the width of a double-stranded α-helical DNA chain (2 nm). The average predicted poly-APS pore size is 2.9 nm, and there may exist larger aggregated pores large enough to allow passage of soluble plasmid cDNA into cells. It could also be that cDNA in solution is capable of intertwining its α-helices to minimise the overall diameter of the two double-stranded DNA chains that are side-by-side. In addition to the size of the pore formed, the transient nature of the pores that have been formed using poly-APS and halitoxin, are likely to be the reason for these reagent's successful use as transfection chemicals. Other pore-forming chemicals such as saponin and α-escin produce pores in plasma membranes large enough to allow passage of sizeable 135-150 kDa proteins. These non-reversible pore-forming chemicals will be adequate for short-term experimentation, but cells exposed to them will eventually die through massive necrosis and Ca²⁺ excitotoxicity. The effective non-reversible nature of such detergents and toxins is what renders them ineffective for DNA transfer in systems where the subsequent prolonged survival of the cell is necessary. The transfer of cDNA into HEK293 cells and subsequent protein expression indicated that both sponge toxin preparations at 0.5 μg/ml were able to provide reversible pores for cDNA transfer without cytotoxicity. The optimisation protocol indicated that pre-incubation with the sponge toxins and pore formation prior to addition of cDNA was useful, suggesting that in solution toxin and cDNA interactions attenuated pore formation and/or cDNA transfer. Similar effects were seen with serum present, which also reduced transfection efficiency achieved with poly-APS. Poly-APS has strong interactions with serum proteins and consistent with this study, incubation of poly-APS with highly diluted serum can prevent the hemolytic activity of poly-APS. Comparison between the methods using lipofectamine and sponge toxin preparations showed that although lipofectamine has higher efficiency, poly-APS has some clear advantages including high stability and good water solubility, and the sponge toxins provide a simpler and more consistent method which are of benefit to in vivo studies.

In summary, the above results show that viable cells transfected with DNA can be obtained using transient pore forming alkyl pyridinium sponge toxins and a simple pre-incubation protocol.

Poly-APS has also been used to deliver the cytoskeletal protein tau in vitro into cultures of hippocampal neurones and in vivo into the rat hippocampus. Hyperphosphorylated tau is a key characteristic of brains from patients with Alzheimer's disease and the possibility of generating models of tauopathies has been investigated. Tau protein induced deficits were evaluated using electrophysiology, Ca²⁺ imaging and water maze learning. The intracellular loading of preparations with the protein was assessed using tau specific antibodies. Cultured hippocampal neurones loaded with tau survived for at least 24 hours and normal action potentials and Ca²⁺ transients could be recorded from them. Similarly in vivo, animals performed water maze learning and memory tests normally after delivery of tau to the hippocampus. However, if in addition to delivering tau, cultures were treated with the phosphatase inhibitor okadaic acid, action potentials and clear Ca²⁺ transients could not be evoked in the few neurones that survived. Furthermore, animals treated in vivo with poly-APS, tau and okadaic acid could learn the water maze platform position, but had impaired reversal learning when the platform position was changed. Long-term potentiation measured in hippocampal slices ex vivo was also attenuated.

Site specific delivery of tau to intracellular compartments followed by hyperphosphorylation provides a new approach to the investigation of cellular aspects of Alzheimer's disease. These data also provide further proof that poly-APS can be used to deliver macromolecules into cells to achieve transfection or model disease states in vitro and in vivo.

Water maze experiments involved training animals to find a platform in a quadrant of a circular bath. Once the animal had learnt the position of the platform the platform could be moved to test the animals ability to show reversal learning.

FIG. 13 shows two trails and the swimming paths taken to the platform. In trail 1 the animal swims round the bath and has not learnt where the platform is but during trial 16 the animal swims directly to the platform.

Poly-APS (sponge toxin) was applied to the hippocampus either alone or with tau, tau was also applied without poly-APS. A mini pump was then used in some animals to deliver okadaic acid over 7 days, this blocks phosphatases resulting in hyperphosphorylation of proteins including tau protein.

In FIG. 14 the data for learning where the platform is for each group of experimental animals is shown. All animals learnt where the platform was, as reflected by the time (above 25%) spent in the quadrant with the platform and the swimming path lengths. When the position of the platform was moved, the animals that had been treated with poly-APS, tau and okadaic acid failed to show learning reversal (FIG. 15). All other treatment groups did.

As can be seen from FIG. 16, which shows ex-vivo data, hippocampal slices were used to study long term potentiation (LTP). LTP is a form of neuronal plasticity which last many hours or days. If pathways get tetanic stimulation (arrow) subsequent electrophysiological signalling is increased. Brains from animals treated with poly-APS, Tau and okadaic acid showed less LTP.

As can be seen from FIG. 17, tau was succesfully delivered to the intracellular compartments of hippocampal neurones in culture. The tau level (black) were detected using specific antibodies to this protein.

As shown in FIG. 18, cultured hippocampal neurones treated with poly-APS and tau produced action potentials, whilst neurones treated with poly-APS, tau and okadaic acid failed to show action potential firing even when stimulated with much larger currents.

As shown in FIG. 19, Cultured hippocampal neurones were stimulated with KCl pulses. Neurones treated with okadaic acid as well as poly-APS and tau failed to show proper calcium transients. With the data shown in FIG. 18, this shows that the excitability of these neurones was inhibited probably by misfunctioning of voltage-activated channels.

As shown in FIG. 20, the mean data for calcium transient for neurones exposed to the different treatments, clearly distinguished the neurones exposed to poly-APS, tau and okadaic acid from the neurones exposed to poly-APS and okadaic acid, and poly-APS and tau. With the data from FIGS. 18 and 19, this shows that the excitability of these neurones was greatly reduced and probably involved misfunctioning of voltage-activated channels.

1-29. (canceled)

30. The composition comprising a sponge toxin for the reversible formation of a membrane pore.

31. The composition according to claim 30, wherein the sponge toxin comprises at least one polymeric 1,3-alkylpyridinium salt (poly-APS).

32. The composition according to claim 30 wherein the sponge toxin is obtained from the group consisting of the sponge Reniera sarai, Callyspongia ridleyi, Haliclona erina, Haliclona rubens, Haliclona viridis, Amphimedon viridis, Callyspongia fibrosa and Amphimedon compressa.

33. The composition according claim 30, wherein the sponge toxin has a molecular weight of between 5 kDa and 20 kDa.

34. The composition according to claim 33, wherein the sponge toxin has a molecular weight of 5.5 kDa or 18.9 kDa.

35. The composition according to claim 30, wherein the concentration of sponge toxin is between 0.5 ng/ml and 5.0 μg/ml.

36. The composition according to claim 35, wherein the concentration of sponge toxin is between 0.5 ng/ml and 0.5 μg/ml.

37. A method for the reversible formation of membrane pores, the method comprising the steps of:
a) incubating the membrane in the presence of a composition according claim 30; and
b) removing the composition from contact with the membrane.

38. The method according to claim 38, further comprising, addition of zinc solution to attenuate the reversible formation of membrane pore.

39. The method according to claim 38 wherein the concentration of zinc solution is between substantially 1 to 2 mM.

40. The method according to claim 39, wherein the concentration of zinc is 1.5 mM.

41. A method for transfection of a macromolecule into a cell in vitro, the method comprising the steps of:
a) incubating the cell in the presence of a composition comprising a sponge toxin;
b) removing the composition from contact with the cell; and
c) adding a macromolecule.

42. The method according to claim 41, wherein the macromolecule is selected from the group consisting of cDNA, protein, peptide, lipid and oligonucleotide.

43. The method according to claim 41, wherein the cell is incubated in the presence of the composition for between 1 and 20 minutes prior to addition of the macromolecule.

44. The method according to claim 43 wherein the cell is incubated in the presence of the composition for 5 minutes prior to the addition of the macromolecule.

45. The method according to claim 42, wherein between 1.0 and 5.0 μg nucleic acid is added.

46. The method according to claim 45, wherein 2.5 μg nucleic acid is added.

47. The method according to claim 41, wherein the cell is incubated in the presence of the composition and macromolecule and the composition and macromolecule are removed and replaced with standard media.

48. The method according to claim 47 wherein the cells are incubated for between 20 and 200 minutes.

49. The method according to claim 48 wherein the cells are incubated for 180 minutes.

50. A method for transfection of a macromolecule into a cell in vivo, the method comprising the step of:
a) incubating the cell in the presence of a composition comprising a sponge toxin and a macromolecule.

51. The method according to claim 50, wherein the macromolecule is selected from the group consisting of cDNA, protein, peptide, lipid and oligonucleotide.

52. The method according to claim 51, wherein the macromolecule is the cytoskeletal protein tau.

53. The method according to claim 50 wherein the cell is a hippocampal neurone.

54. A model for use in the study of neurological disease or treatments thereof, the model comprising a rodent having undergone application of a composition comprising a sponge toxin, tau protein and phosphatase inhibitor to the hippocampus.

55. The model according to claim 54 wherein the neurological disease is Alzheimer's disease.

56. The model according to claim 54 wherein the rodent is a rat or a mouse.

57. The model according to claim 54 wherein the phosphatase inhibitor is okadaic acid.

58. A method of studying a neurological disease, the method comprising:
a) applying a composition comprising a sponge toxin, tau protein and phosphatase inhibitor to the hippocampus of a rodent; and
b) studying the effect on the rodent.

59. The method according to claim 58 wherein the phosphatase inhibitor is okadaic acid.

60. The composition according to claim 31, wherein the sponge toxin has a molecular weight of between 5 kDa and 20 kDa.

61. The composition according to claim 60, wherein the sponge toxin has a molecular weight of 5.5 kDa or 18.9 kDa.

62. The composition according to claim 31, wherein the concentration of sponge toxin is between 0.5 ng/ml and 5.0 μg/ml.

63. The composition according to claim 62, wherein the concentration of sponge toxin is between 0.5 ng/ml and 0.5 μg/ml.
